(19) United States
(12) Patent Application Publication
(10) Pub. No.: US 2022/0370804 A1
(43) Pub. Date: Nov. 24, 2022

(54) MOTOR LEARNING AND VAGUS NERVE STIMULATION (VNS) PAIRED WITH MOTOR LEARNING TO TREAT DEMYELINATING DISEASES, CONDITIONS AND DISORDERS

(71) Applicant: THE REGENTS OF THE UNIVERSITY OF COLORADO, A BODY CORPORATE, Denver, CO (US)

(72) Inventors: Cristin Welle, Denver, CO (US); Ethan Hughes, Denver, CO (US); Rongchen Huang, Fort Collins, CO (US)

(21) Appl. No.: 17/581,307

(22) Filed: Jan. 21, 2022

Related U.S. Application Data
(60) Provisional application No. 63/140,686, filed on Jan. 22, 2021.

(51) Int. Cl. A61N 1/36

(52) U.S. Cl. CPC A61N 1/36053; A61N 1/3606; A61N 1/36157; A61N 1/36034; A61N 1/36025; A01K 29/005

(57) ABSTRACT

Embodiments of the instant invention relate to applying motor learning to promote remyelination following demyelination in a subject having a condition or disease. In certain embodiments, applying motor learning alone or in combination with vagus nerve stimulation (VNS) induces the production of new and preserves surviving oligodendrocytes. In accordance with certain embodiments of the disclosure, motor learning, when properly timed, enhances oligodendrogenesis after injury and recruits mature oligodendrocytes to participate in remyelination through the generation of new myelin sheaths. In other aspects of the disclosure, VNS paired with motor learning enhances remyelination following demyelination.

### PRIORITY

This U.S. Non-Provisional Application claims priority to U.S. Provisional Patent Application No. 63/140,686 filed Jan. 22, 2021. This provisional application is incorporated by reference in its entirety for all purposes.

### GOVERNMENT FUNDING

This invention was made with government support under grant number HR0011-17-2-0051 awarded by DARPA. The government has certain rights in the invention.

### FIELD

Embodiments of this disclosure relate to systems and methods for treating or ameliorating symptoms or side effects of demyelinating diseases, conditions, and disorders. In certain embodiments, systems and methods disclosed herein use mechanisms and processes for vagus nerve stimulation to treat, prevent or ameliorate demyelination diseases, conditions or symptoms.

### BACKGROUND

A variety of central nervous system (CNS) demyelinating disorders, including multiple sclerosis (MS), acute disseminated encephalomyelitis and neuromyelitis optica spectrum disorders, are difficult to effectively treat. For example, multiple sclerosis (MS) is a neurodegenerative and neuroinflammatory disease that damages oligodendrocytes, leading to demyelination and eventual degeneration and loss of axons. Although the root cause of demyelination is not well understood, it is generally associated with the formation of lesions on the myelin sheaths and inflammation. Current treatments, with modest to little success, are primarily directed to treating acute attacks and reducing the frequency of attacks in the relapsing-remitting subtype of the disease or treating the symptoms. However, current therapies appear to only slow the progression of the disease.

Remyelination is a potential and promising approach to treat MS, as it prevents axonal degeneration and restores functional neural activity. However, spontaneous remyelination is often incomplete, often resulting in permanent motor cognitive deficits in patients suffering from these conditions. Accordingly, there is a need for identifying effective strategies to enhance remyelination to preserve and restore function.

### SUMMARY

Embodiments of the instant invention relate to systems and methods for treating or ameliorating symptoms or side effects of demyelinating diseases, conditions, and disorders. In certain embodiments, systems and methods disclosed herein use mechanisms and processes of motor stimulation in order to stimulate vagus nerves to treat, prevent or ameliorate demyelination diseases, conditions or symptoms.

In some embodiments, systems and methods including treating a subject having a demyelinating disease, condition or disorder by introducing and using motor learning or motor learning skills suppresses oligodendrogenesis while increasing oligodendrocyte generation, OPC differentiation, and/or retraction of pre-existing myelin sheaths leading to an improved condition in the subject. In some embodiments, introducing and using motor learning or motor learning skills in a subject in need thereof induces remyelination of the subject and improvement of the disease or condition. In certain embodiments, motor learning or motor learning skills is required once per day or multiple times per day in order to treat the condition or disease in a subject in need thereof. In certain embodiments, motor learning or motor learning skills when introduced to a subject in need thereof reduce progression and/or reverse demyelination in the subject.

In some embodiments, the subject in need of such a treatment has a demyelination condition. In other embodiments, the subject in need of such a treatment has a demyelination disease. In yet other embodiments, the subject in need of such a treatment has experienced an acute injury causing demyelination. In certain embodiments, a condition to be treated in a subject disclosed herein include, but is not limited to, a central nervous system (CNS) demyelinating disorder. In accordance with these embodiments, a CNS demyelinating disorder includes, but is not limited to, multiple sclerosis (MS), acute disseminated encephalomyelitis and neuromyelitis optica spectrum disorders. In some embodiments, a subject to be treated by systems and methods disclosed herein has multiple sclerosis (MS). In accordance with these embodiments, the subject having MS is experiencing a neurodegenerative and neuroinflammatory disease that damages oligodendrocytes, leading to demyelination and eventual degeneration and loss of axons. Systems and methods disclosed herein can reduce or prevent formation of lesions on the myelin sheaths and reduce inflammation. In other embodiments, the subject has another demyelinating disorder or condition.

### BRIEF DESCRIPTION OF THE DRAWINGS

FIGS. 1A-1K: FIG. 1A illustrates a mouse performing a motor learning task; FIG. 1B outlines multiple motor learning task schedules; FIG. 1C provides images of mouse neurons at multiple times relative to a motor learning task regimen; FIGS. 1D, 1E, 1F, and 1G summarize nerve development in mice subjected to motor learning task regimens; FIG. 1H provides images of developing myelin sheaths in mice subjected to motor learning tasks; and FIGS. 1I, 1J, and 1K summarize myelin sheath development in mice subjected to motor learning tasks in accordance with certain embodiments of the invention.

FIGS. 2A-2G: FIG. 2A provides neural images of mice subjected to motor learning tasks. FIG. 2B outlines a motor learning task schedule; FIG. 2C provides a summary of nerve cell differentiation rates as a function of time relative to motor learning task participation; FIG. 2D provides a summary of nerve cell proliferation rates as a function of time relative to motor learning task participation; FIG. 2E provides a summary of nerve cell death as a function of time relative to motor learning task participation; FIG. 2F provides prevalences for differentiation and proliferation in demyelinated nerve cells; and FIG. 2G provides prevalences for differentiation and proliferation in demyelinated nerve cells as a function of time relative to motor learning task participation in accordance with certain embodiments of the invention.

FIGS. 3A-3J: FIG. 3A outlines a treatment regime including demyelination and subsequent remyelination; FIG. 3B provides brain images during multiple stages of demyelination and remyelination; FIG. 3C provides a comparison of nerve cell lifespans during demyelination and remyelination; FIG. 3D provides myelin images during demyelination and remyelination periods; FIG. 3E provides timelines for nerve degeneration during and after demyelination. FIGS. 3F, 3G summarize nerve growth and loss during and after demyelination; FIG. 3H provides a comparison of lost and replaced nerve cells during and after demyelination; FIG. 3I summarizes neuron firing rates in healthy and remyelinating mice; and FIG. 3J provides a comparison of neuron firing rates during and after demyelination in accordance with certain embodiments of the invention.

FIGS. 4A-4I: FIG. 4A provides brain images during multiple stages of myelination and demyelination; FIG. 4B provides a summary of nerve growth at multiple timepoints during and after demyelination; FIG. 4C provides a summary of myelin development at multiple timepoints during and after demyelination; FIG. 4D provides a comparison of myelination in growing and retracting neurons; FIGS. 4E and 4F provide an overview of myelin sheath length in developing neurons; FIG. 4G displays myelin prevalence in developing neurons; FIG. 4H displays images of developing neurons in multiple, differently myelinated neural tissues; and FIG. 4I provides a comparison of myelin development across multiple neural tissues in accordance with certain embodiments of the invention.

FIGS. 5A-5N: FIG. 5A outlines multiple demyelination and motor learning task schedules; FIG. 5B summarizes nerve cell development as a function of demyelination and motor learning task regimen; FIG. 5C summarizes nerve cell loss over multiple demyelination and motor learning task regimens; FIG. 5D summarizes nerve cell development over multiple demyelination and motor learning task regimens; FIG. 5E provides a summary of motor learning task performance during multiple demyelination and motor learning task regimens; FIG. 5F provides a summary of neural activity during multiple demyelination and motor learning task regimens; FIG. 5G summarizes motor learning task success rates during multiple demyelination and motor learning task regimens; FIG. 5H provides a summary of nerve cell development over multiple demyelination and motor learning task regimens; FIG. 5I summarizes nerve cell generation over multiple demyelination and motor learning task regimens; FIG. 5J provides a summary of motor learning task performance during multiple demyelination and motor learning task regimens; FIG. 5K provides a summary of neural activity during multiple demyelination and motor learning task regimens; FIG. 5L provides a summary of motor learning task performance during multiple demyelination and motor learning task regimens; FIG. 5M summarizes nerve cell development over multiple demyelination and motor learning task regimens; and FIG. 5N summarizes nerve cell generation over multiple demyelination and motor learning task regimens in accordance with certain embodiments of the invention.

FIGS. 6A-6I: FIG. 6A provides brain images over multiple demyelination and motor learning task regimens; FIG. 6B summarizes nerve cell generation over multiple demyelination and motor learning task regimens; FIG. 6C summarizes neural tissue densities during following demyelination as a function of motor learning task participation; FIG. 6D summarizes myelin development as a function of motor learning task participation; FIG. 6E provides a comparison of myelin development in growing and retracting nerve cells as a function of motor learning task participation; FIGS. 6F and 6G detail remyelination as a function of motor learning task participation; FIG. 6H summarizes myelin development following demyelination as a function of motor learning task participation; and FIG. 6I compares remyelination of denuded nerve cells as a function of motor learning task participation in accordance with certain embodiments of the invention.

FIGS. 7A-7O: FIGS. 7A, 7B and 7C provide mouse brain images highlighting myelination and cell survival following demyelination; FIG. 7D summarizes myelin growth and loss in nerve cells following demyelination; FIG. 7E overviews myelin growth in nerve cells following demyelination; FIG. 7F provides a summary of nerve cell survival following demyelination; FIG. 7G provides a summary of myelin development as a function of motor learning task participation; FIG. 7H provides a summary of myelin development at multiple time points following demyelination; FIGS. 7I and 7J provide summaries of myelin loss as a function of motor learning task participation; FIG. 7K provides brain images following demyelination for mice which did not partake in motor learning tasks; FIG. 7L overviews myelin development as a function of motor learning task participation; FIG. 7M provides images of nerve cells in multiple, differently myelinated brain regions; FIG. 7N provides a comparison of myelin development in nerve cells which survived or developed subsequently to a demyelination event; and FIG. 70 details myelination as a function of motor learning task participation in accordance with certain embodiments of the invention.

FIGS. 8A-8F: FIGS. 8A-8B summarize nerve cell generation following demyelination as a function of motor learning task participation and vagus nerve stimulation; FIG. 8C summarizes lost nerve cell replacement as a function of motor learning task participation and vagus nerve stimulation; FIGS. 8D-8E provide rates of lost nerve cell replacement as a function of motor learning task participation and vagus nerve stimulation; and FIG. 8F summarize nerve cell growth following demyelination as a function of motor learning task participation and vagus nerve stimulation in accordance with certain embodiments of the invention.

FIGS. 9A-9C: FIGS. 9A-9B detail lost nerve cell replacement as a function of motor learning task participation and vagus nerve stimulation; and FIG. 9C summarizes rates of lost nerve cell replacement as a function of motor learning task participation and vagus nerve stimulation.

FIGS. 10A-10B: FIG. 10A overviews motor learning task performance for vagus nerve stimulated and unstimulated mice; and FIG. 10B summarizes improvements in motor learning task performance as a function of vagus nerve stimulation in accordance with certain embodiments of the invention.

FIGS. 11A-11C: FIG. 11A provides a comparison of motor learning task performance by learning motivated and unmotivated mice; and FIGS. 11B-11C summarizes improvements in motor learning task performance by multiple mice in accordance with certain embodiments of the invention.

FIGS. 12A-12F: FIG. 12A provides multiple types of nervous tissue images; FIG. 12B myelin sheath detection with different imaging techniques; FIG. 12C provides images showing maximum myelin resolution achieved with multiple techniques; FIG. 12D summarizes myelin sheath lengths observed with different imaging techniques; and FIGS. 12E-12F provides images of myelin generated with multiple imaging techniques in accordance with certain embodiments of the invention.

FIGS. 13A-13J: FIG. 13A provides a schematic for nerve cell development; FIG. 13B details nerve cell development in mice over ten weeks; FIG. 13C provides a summary of nerve cell development as a function of motor learning task participation; FIG. 13D provides a summary of nerve cell development as a function of diet and motor learning task participation; FIG. 13E provides a comparison of motor learning task performance and nerve development; FIG. 13F provides a summary of nerve cell development rates as a function of motor learning task participation; FIGS. 13G-13H provide nerve cell proliferation rates a function of time relative to participation in a motor learning task; FIG. 13I summarizes nerve cell migration in developing neural tissue; and FIG. 13J summarizes nerve cell migration prevalency as a function of time relative to participation in a motor learning task in accordance with certain embodiments of the invention.

FIGS. 14A-14J: FIG. 14A provides images of brains prior to and following demyelination; FIG. 14B summarizes demyelination effects on myelin and nerve cell densities; FIG. 14C provides images of brains collected with multiple imaging methods; FIG. 14D overviews effects of demyelination on multiple types of nerve cells; FIG. 14E provides images with maximal nerve cell resolution achieved with multiple imaging methods; FIG. 14F provides a summary of effects of demyelination on multiple types of nerve cells; FIG. 14G provides images of brains prior to and following demyelination generated with multiple imaging techniques; FIG. 14H provides images with maximal nerve cell resolution obtained with multiple imaging methods and at different times relative to demyelination; FIG. 14I compares nerve cell resolution achieved with multiple imaging methods; and FIG. 14J summarizes nerve cell densities resolved with multiple imaging methods in accordance with certain embodiments of the invention.

FIGS. 15A-15M: FIG. 15A provides a summary of nerve cell loss as a function of tissue depth and time relative to demyelination; FIG. 15B provides a summary of nerve cell gain as a function of tissue depth and time relative to demyelination; FIG. 15C provides a comparison of nerve cell survival and death for demyelinated and non-demyelinated nerve cells; FIG. 15D provides a comparison of nerve cell gain and loss during demyelination; FIG. 15E provides a summary of tissue depth for nerve cells generated during demyelination; FIG. 15F nerve growth rate during demyelination and motor learning task events; FIG. 15G summarizes nerve cell gain during demyelination; FIG. 15H summarizes nerve cell loss during demyelination. FIG. 15I summarizes nerve cell replacement during demyelination; FIG. 15J depicts nerve cells proximal to an electrode and containing varying degrees of myelination; FIG. 15K provides images of myelinated (left) and unmyelinated (right) nerve cells; FIG. 15L provides a comparison of myelination in nerve cells subjected and not subjected to a demyelination regimen; and FIG. 15M provides a comparison of predicted and observed nerve cell myelination frequencies following demyelination in accordance with certain embodiments of the invention.

FIGS. 16A-16L: FIG. 16A provides a timeline for motor learning task intervention following demyelination; FIG. 16B provides a comparison of attempts to complete motor learning tasks as a function of demyelination; FIG. 16C provides a summary of motor learning task success rates as a function of demyelination; FIG. 16D details motor learning task improvement as a function of demyelination; FIG. 16E provides a summary of motor learning task performance as a function of nerve cell loss; FIG. 16F provides a summary of motor learning task performance as a function of nerve cell replacement; FIG. 16G provides a timeline for performing a motor learning task following demyelination; FIG. 16H provides a comparison of attempts to complete motor learning tasks as a function of demyelination; FIG. 16I provides a summary of motor learning task success rates as a function of demyelination; FIG. 16J details motor learning task improvement as a function of demyelination; FIG. 16K provides a summary of motor learning task performance as a function of nerve cell loss; and FIG. 16L provides a summary of motor learning task performance as a function of nerve cell replacement in accordance with certain embodiments of the invention.

FIGS. 17A-17I: FIG. 17A provides a timeline for motor learning task participation prior to and following demyelination; FIG. 17B provides a summary of motor learning task performance as a function of demyelination; FIG. 17C outlines nerve replacement during motor learning task participation following demyelination; FIG. 17D provides a summary of nerve replacement rates following demyelination as a function of motor learning task participation; FIG. 17E provides a summary of motor learning task activity as a function of demyelination; FIG. 17F provides a summary of motor learning task performance as a function of time and demyelination; FIG. 17G overviews changes in motor learning task performance as a function of demyelination; FIG. 17H provides a comparison of motor learning task performance and nerve cell loss during demyelination; and FIG. 17I provides a comparison of nerve cell replacement and motor learning task performance in accordance with certain embodiments of the invention.

FIGS. 18A-18E: FIG. 18A provides images of nerve cells before and after demyelination; FIG. 18B details changes in nerve cell morphology following demyelination; FIG. 18C provides a comparison of nerve cell sizes during and after demyelination; FIG. 18D summarizes changes in nerve cell size during and after demyelination; and FIG. 18E provides an overview of myelin development in neural tissue before, during, and after demyelination in accordance with certain embodiments of the invention.

FIGS. 19A-19J: FIG. 19A provides an overview of cell survival as a function of motor learning task participation; FIG. 19B provides an overview of cell survival following demyelination as a function of motor learning task participation; FIG. 19C provides a summary of myelin loss as a function of motor learning task participation; FIG. 19D provides a summary of myelin growth as a function of motor learning task participation; FIG. 19E provides images neurons over multiple periods of a motor learning task regimen; FIG. 19F provides a summary of myelin retention for growing and retracting neurons as a function of demyelination; FIG. 19G provides a summary of myelin retention as a function of motor learning task participation and demyelination; FIG. 19H provides images of newly generated myelin following demyelination. FIG. 19I provides a summary of myelin growth as a function of days since birth; and FIG. 19J provides a summary of myelin growth for growing and retracting nerve cells in accordance with certain embodiments of the invention.

FIGS. 20A-20C: FIG. 20A provides images of nerve cells collected with multiple imaging techniques; FIG. 20B provides a summary of changes in nerve cell volume as a function of demyelination; and FIG. 20C provides a summary of size change for demyelinated nerve cells in accordance with certain embodiments of the invention.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject” can refer to any mammal, including but not limited to, a non-human primate (for example, a monkey or great ape), livestock or pets such as a cow, a pig, a cat, a dog, a rat, a mouse, a horse, a goat, a rabbit, a sheep, a hamster, a guinea pig) or other subject. In some embodiments, the mammalian subject is a human such as an adult, a young child, adolescent, toddler, or infant.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that times and other specific details can be modified through routine experimentation. In some cases, well known methods, or components have not been included in the description.

In certain embodiments, motor learning has been identified to promote remyelination following demyelination in a subject due to a condition or injury. In some embodiments, motor learning skills can be used to generate new and for protecting surviving oligodendrocytes. In accordance with certain embodiments of the disclosure, motor learning as disclosed herein, when appropriately timed with respect to an injury, insult or onset of a condition, enhances oligodendrogenesis and recruits mature oligodendrocytes to participate in remyelination through the generation of new myelin sheaths and/or preserving myelin sheaths from destruction or injury. In other embodiments, systems and methods using vagus nerve stimulation (VNS) paired with motor learning can induce or enhance remyelination following demyelination. In accordance with these embodiments, following demyelination, VNS paired with learning of a skilled motor task leads to increased remyelination over both subjects learning the skilled motor task without VNS and over subjects receiving VNS without learning a skilled motor task. In certain embodiments, skilled motor task can be used to improve outcome of a subject having a demyelination injury or condition. In certain embodiments, a temporally precise combination of VNS applied during motor skill learning can optimize remyelination instead of using either method alone. In some embodiments, the combination of VNS and learning a skilled motor task in a subject in need thereof is synergistic compared to using either method alone.

It is understood by one of skill in the art that proper tissue regeneration following injury or disease is a long sought-after goal of medical professional, particularly in the adult nervous system. Oligodendroglia represent one of the few cell types that retain the capacity to regenerate and repair following damage to the adult CNS. Remyelination of denuded axons can restore neuronal function, promote neuroprotection, and can facilitate functional recovery in CNS diseases, conditions or injury characterized by myelin loss.

As disclosed herein, embodiments include using motor learning skills to improve outcome and recovery in a subject having a CNS disease, condition or injury characterized by myelin loss. It was demonstrated that learning shapes the pattern of myelination in the healthy and remyelinating brain. Longitudinal in vivo two-photon imaging of the forelimb motor cortex throughout the learning of a forelimb reach task revealed that learning suppressed oligodendrogenesis but subsequently increased oligodendrocyte generation, OPC differentiation, and retraction of pre-existing myelin sheaths. In some embodiments, motor learning applied to subject led to remyelination and restoration of neuronal function and resulted in greater oligodendrocyte and myelin sheath replacement. Additionally, motor learning enhanced the ability of surviving oligodendrocytes to participate in remyelination via the generation of new sheaths. These results demonstrate that motor learning can improve remyelination via cortical oligodendrogenesis and myelin sheath formation by surviving oligodendrocytes.

Discoveries disclosed herein are the first to observe a learning-induced suppression in oligodendrocyte generation and in certain cases, a transient learning-induced suppression. OPC differentiation was unaffected during this suppression, suggesting that learning can temporarily decrease the survival and integration of differentiated OPCs as mature myelinating oligodendrocytes. It is possible that location-specific cues suppress the integration of new oligodendrocytes to prevent aberrant myelination during learning, or that metabolic demand imposed by network plasticity during learning can deplete the resources required for the generation and integration of adjacent oligodendrocytes. Axons form synapses with local OPCs, and neuronal activity can modulate OPC proliferation and differentiation within both healthy CNS and demyelinated regions. This communication can be mediated by effects of brain-derived neurotrophic factor on both activity-dependent synaptic modulation and oligodendrocyte maturation and myelination.

Oligodendrocytes, the myelin-forming cells of the central nervous system (CNS), enhance the propagation of action potentials and support neuronal and axonal integrity through metabolic coupling. Injury to oligodendrocytes critically affects axonal health and is associated with significant neurologic disability, e.g., in patients with multiple sclerosis (MS). Oligodendrocyte precursor cells (OPCs) can generate new oligodendrocytes with the capacity to remyelinate denuded axons, which can restore neuronal function. However, remyelination is typically incomplete in patients with MS, and approaches to increase myelin repair remain limited. Embodiments disclosed herein have demonstrated that remyelination can occur using a treatment regimen including applying motor learning skills with or without VNS in a subject having MS to induce remyelination of denuded axons and restore neuronal function in the subject.

Vegas Nerve Stimulation (VNS) has been implicated to drive cortical neural activity, which plays a role in remyelination. Further, pairing vagus nerve stimulation with rehearsal motor behavior (e.g., paired VNS with rehabilitation training) has been implicated to enhance cortical plasticity and improve motor performance.

Motor learning drives white matter changes in mammals (e.g. humans) in part by eliciting the proliferation and differentiation of OPCs in the adult CNS similar to OPC responses to demyelinating injuries, yet it remains unclear whether learning during demyelination has synergistic or antagonistic effects. Behavioral interventions are increasingly personalizable in clinical settings and are used to ameliorate motor function in myelin disease patients. Optimizing the modality and timing of behavioral interventions can allow endogenous mechanisms of myelin plasticity to act in synchrony and drive more robust remyelination following injury.

In certain embodiments, it was discovered that motor learning in a subject having a demyelination disorder promotes remyelination following demyelination via new and surviving oligodendrocytes. In accordance with certain embodiments of the disclosure, it has been found that motor learning, when properly timed, enhances oligodendrogenesis after injury and recruits mature oligodendrocytes to participate in remyelination through the generation of new myelin sheaths.

In accordance with certain embodiments of the disclosure, through longitudinal in vivo two-photon imaging of oligodendrocyte lineage cells and individual myelin sheaths, the complex dynamics between motor skill acquisition and oligodendroglia in the motor cortex have been defined. Certain embodiments relate to both developmental and remyelinating contexts using a demyelination model, which results in ongoing oligodendrocyte death and regeneration (similar to cortical lesions in MS patients without the confound of autoimmunity).

Some embodiments of the instant disclosure relate to methods for preventing demyelination, reducing demyelination, promoting remyelination, or a combination thereof, in a subject having a demyelination disease, disorder, or condition. In certain aspects, the methods involve having a subject perform motor learning tasks. In other aspects, the methods involve stimulation of vagus nerve fibers in a subject paired with performance of motor learning tasks. In other embodiments, performing at least one motor learning task by a subject contemplated herein includes, but is not limited to, performing at least one motor learning task: multiple times per day, twice per day, daily, every other day, a couple times a week or other appropriate regimen.

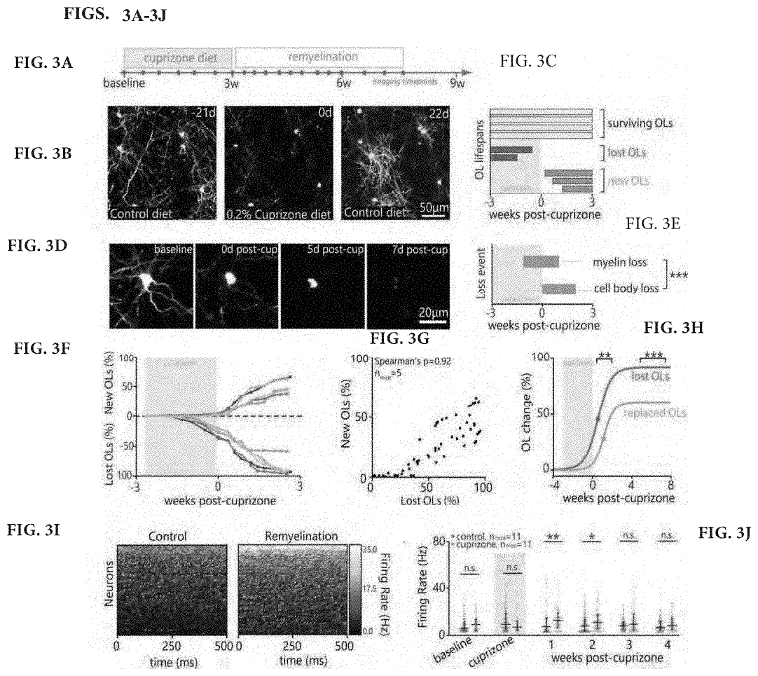

In certain embodiments, the present disclosure relates to one surprising discovery that delayed onset of motor learning tasks can affect remyelination and cognitive and motor function recovery following traumatic injury (e.g., a brain injury, accident or a concussion). As disclosed herein (see for example, FIGS. 3A-3J) oligodendrocyte and myelin formation can be delayed following a demyelination event, such as those affected by concussions. Accordingly, a motor learning task may not be optimally effective in initiating myelination responses when performed close to a demyelination events, or during periods of active remyelination. It is also noted that competency gained during early motor learning task performance may diminish effects of motor learning task performance during later periods, when neural tissue may otherwise be more responsive to such regimens. In certain embodiments, methods disclosed herein concern first performing a motor learning task at least 2 days, at least 3 days, at least 5 days, at least 7 days, at least 10 days, at least 14 days, at least 21 days, at least 28 days, at least 40 days, or at least 60 days after a traumatic injury can improve outcome of a subject in need of such a treatment such as subject suffering from a traumatic brain injury (TBI) or concussion. In some embodiments, methods disclosed herein concern performing a motor learning task at least 7 days after a traumatic injury. In other embodiments, methods disclosed herein include first performing a motor learning task at least 14 days after a traumatic injury. In yet other embodiments, methods of the present disclosure include first performing a motor learning task at least 28 days after a traumatic injury.

In certain embodiments, motor learning task efficacy can be enhanced by intermittency. For example, a break of at least 1 day, at least 2 days, at least 3 days, at least 5 days, at least 7 days, at least 10 days, at least 14 days, at least 21 days, at least 28 days, at least 40 days, or at least 60 days to enhance remyelination or improve cognitive or motor function. In other embodiments, methods disclosed herein concern a subject having a TBI ceasing motor learning tasks for at least 7 days before resuming a motor learning task regimen. In other embodiments, a subject having a TBI can perform multiple motor learning tasks in succession for a single session or multiple sessions prior to or following an interruption or break from motor task learning.

In certain embodiments, a subject can be exposed to a single motor learning task or multiple learning tasks if determined to be more effective than a single task. In other embodiments, motor learning tasks include more than one task alone or in combination with VNS. In other embodiments, a single motor learning task can be applied to a subject having a demyelination condition in order to simplify the process, improve efficiency and/or ensure compliance for improved results.

In other embodiments, methods include using dietary restrictions to enhance treatment methods for a subject in need of treatments disclosed herein. In some embodiments, restricting a subject's diet enhance therapeutic benefits of motor learning tasks to reduce demyelination and/or enhance remylination. In certain embodiments, dietary restrictions include reducing caloric intake. In accordance with these embodiments, reduced caloric intake prior to, during, and after motor learning task participation can augment increases in nerve cell and myelin generation. In some embodiments, a methods include reducing a subject's caloric intake by at least about 5%, at least about 10%, at least about 20%, at least about 30%, at least about 40%, or at least about 50%. In other embodiments, methods can include restricting a subject's caloric intake to a maximum of about 2500 calories per day, a maximum of about 2000 calories per day, a maximum of about 1800 calories per day, a maximum of about 1500 calories per day, a maximum of about 1200 calories per day, or other appropriate level. In other embodiments, caloric intake can be varied during the times when treatment occurs such as every other day or other regimen. In certain embodiments, caloric intake restriction can be prior to, during, or subsequent to, participation in a motor learning task. In certain embodiment, caloric intake restriction is concurrent with the motor learning task. In other embodiments, caloric intake can be reduced in a subject for at least 3 days, at least 5 days, at least 7 days, at least 10 days, at least 14 days, at least 21 days, at least 28 days, at least 40 days, at least 60 days, at least 3 months, at least 6 months, or at least 1 year or more depending on need. In other embodiments, the caloric intake restriction can span the entirety of a motor learning task regimen. In one embodiment, a two-week motor learning task regimen with alternating motor learning task participation and rest days can be paired with a two-week caloric intake restriction. In other embodiments, methods can further include vagus nerve stimulation.

In some embodiment, the dietary restriction include reducing inflammatory food consumption in a restricted caloric intake diet or a special diet to reduce inflammatory contributing foods and/or beverages. Certain refined carbohydrates (such as white breads and processed sugars), meats, trans fats (e.g., many partially hydrogenated oils), omega-6 containing foods, and nightshade family vegetables can affect inflammatory responses, which in turn can inhibit nerve cell development. Accordingly, a dietary restriction can include reducing or eliminating inflammatory food consumption. Such a dietary restriction can include, but is not limited to, limiting inflammatory food consumption to no more than about 400 grams per day (by total food weight), no more than about 200 grams per day, no more than about 100 grams per day, no more than about 80 grams per day, no more than about 60 grams per day, or no more than about 40 grams per day. A dietary restriction can include limiting inflammatory food consumption to a maximum of about 1000 calories per day, a maximum of about 800 calories per day, a maximum of about 600 calories per day, a maximum of about 400 calories per day, a maximum of about 200 calories per day, or a maximum of about 100 calories per day.

In certain embodiments, the methods, devices and systems disclosed herein can be applied specifically to treat any disorder for which a prevention or reduction of demyelination and/or a promotion or increase in remyelination would be beneficial. In accordance with embodiments of the disclosure, demyelination diseases, disorders and conditions which can be treated using the methods, devices and systems as described herein include diseases, disorders, and conditions involving demyelinated nerves, including neuroinflammatory disorders and neuropathies. By way of example, demyelination diseases, disorders, and conditions include, but are not limited to, multiple sclerosis (MS), Alzheimer's disease, Parkinson's disease, Huntington's disease, Amyotrophic lateral sclerosis (ALS), chronic inflammatory demyelinating polyneuropathy (CIDP), and Batten disease; neuroinflammatory disorders, including but not limited to acute disseminated encephalomyelitis (ADEM), acute optic neuritis (AON), transverse myelitis, and Neuromyelitis optica spectrum disorders (NMO); traumatic brain injury, side effects of a brain injury, accident or a concussion and neuropathies, including but not limited to peripheral neuropathies, cranial neuropathies, and autonomic neuropathies.

In some embodiments, the subject has a demyelination disease, disorder, or condition and has experienced demyelination of one or more nerve fibers. In other embodiments, the methods include performing a motor learning task after demyelination has occurred (referred to herein as “delayed learning”). In certain embodiments, methods can include stimulation of the vagus nerve fibers as needed, e.g., when the subject is at an increased risk for demyelination and/or is experiencing (or has experienced) demyelination. Further, the methods can include stimulation of the vagus nerve fibers during or after successful learning of a skilled motor task (referred to herein as “reinforcement stimulation paired with motor learning outcome success”).

In some embodiments, methods disclosed herein can include, but are not limited to, stimulation of the vagus nerve. In certain embodiments, vagus nerve stimulation involves the use of a device to stimulate the vagus nerve with electrical impulses. An implantable vagus nerve stimulator is currently FDA-approved to treat epilepsy and depression and can be used for diseases and conditions disclosed herein. There is one vagus nerve on each side of your body, running from your brainstem through your neck to your chest and abdomen. In conventional vagus nerve stimulation, a device can be surgically implanted under the skin on your chest, and a wire can be threaded under your skin connecting the device to the left vagus nerve. When activated, the device sends electrical signals along the vagus nerve to the subject's brainstem, which then sends signals to certain areas in the subject's brain. Of note, the right vagus nerve isn't typically used because it's more likely to carry fibers that supply nerves to the heart.

In certain embodiments, noninvasive vagus nerve stimulation (VNS) devices, which don't require surgical implantation, can be used in systems and methods disclosed herein. In one embodiment, a noninvasive device that stimulates the vagus nerve can be used to treat subject disclosed herein that has been successfully used for cluster headaches. In certain embodiments, VNS stimulating devices can be either through an invasive implanted stimulation device, or through non-invasive stimulation device, e.g., worn on the ear. For example, the methods, devices and systems for VNS can include, but are not limited to, electrodes (e.g., cuff electrodes, microstimulators) that can be placed around the vagus nerve and can communicate with one or more stimulators configured to apply appropriate stimulation of the vagus nerve to modulate demyelination and/or remyelination. In certain embodiments, the stimulator can be implanted. In other embodiments, the stimulator is integral to the electrodes and can be charged externally.

In some embodiments, the vagus nerve stimulating device can be non-invasive. For example, the device can be worn outside the body and can trigger stimulation of the vagus nerve from a site external to the body (e.g., ear, neck, torso, etc.). In certain embodiments, a non-invasive device can include a mechanical device (e.g., configured to apply vibratory energy to stimulate the vagus nerve of the subject). In some embodiments, the device can be configured to apply ultrasound that can specifically target the vagus nerve and apply energy to activate the vagus nerve. In some embodiments, transcutaneous magnetic stimulation of the vagus nerve can be used. In certain embodiments, VNS stimulation can be combined with using at least one learned motor skill to treat a condition in a subject.

In some embodiments, the vagus nerve can be stimulated for a duration of less than 20 minutes, less than 15 minutes, less than 10 minutes, less than 5 minutes, less than 2 minutes, less than 1 minute, less than 30 seconds or less than 15 seconds. In certain embodiments, the vagus nerve can be stimulated for a duration of at least 15 seconds, at least 30 seconds, at least 1 minute, at least 2 minutes, at least 5 minutes, at least 10 minutes, at least 15 minutes, at least 20 minutes, at least 30 minutes, or at least 1 hour. In certain embodiments, the vagus nerve can be stimulated constantly over a pre-determined time-period. In other embodiments, the vagus nerve can be stimulated intermittently over a pre-determined time-period. In some embodiments, the applied energy to stimulate the vagus nerve can be electrical energy that is a fixed current having a frequency current that is within the range of about 0.05 mA to about 100 milliamperes (mA), about 0.5 to about 20 mA, about 2 to about 50 mA, about 10 to about 50 mA, or about 20 to about 60 mA. In other embodiments, the electrical energy can be at a frequency of about 0.3 to about 3000 Hz, about 1 Hz to about 1000 Hz, about 3 to about 300 Hz, about 1 Hz to about 100 Hz, about 1 Hz to about 30 Hz, or about 10 Hz to about 200 Hz. The pulses can have a width of about 1 to about 1000 μs (e.g., a 200 μs pulse), about 5 to about 200 μs, about 10 to about 100 μs, or about 20 to about 50 μs.

In some embodiments, a device or system for modulating demyelination and/or remyelination can include a stimulator element (e.g., an electrode, actuator, etc.) and a controller for controlling the application of stimulation by the stimulator element. In some embodiments, a stimulator element can be configured for electrical stimulation (e.g., an electrode such as a cuff electrode, needle electrode, paddle electrode, non-contact electrode, array or plurality of electrodes, etc.), mechanical stimulation (e.g., a mechanical actuator, such as a piezoelectric actuator or the like), ultrasonic actuator, thermal actuator, or the like.

In some embodiments, the systems and/or devices of use to stimulate the left vagus nerve are implantable. In other embodiments, the systems and/or device are non-invasive. In general, a controller can include control logic (hardware, software, firmware, or the like) to control the activation and/or intensity of the stimulator element. The controller can control the timing (e.g., on-time, off-time, stimulation duration, stimulation frequency, etc.). In embodiments in which the applied energy is electrical, the controller can control the applied waveform (amplitude, frequency, burst duration/inter-burst duration, etc.). Other components can also be include as part of any of these device or system, such as a power supply (e.g., battery, inductive, capacitor, etc.), transmit/receive elements (e.g., antenna, encoder/decoder, etc.), signal generator (e.g., for conditioning or forming the applied signal waveform), and the like. In some embodiments, a rechargeable battery that can be inductively charged allows the stimulator to deliver numerous electrical stimulations before needing to be recharged. In other embodiments, one or more capacitors that can also be inductively charged can be used to store a limited amount of energy that can be sufficient to deliver a single stimulation or a daily amount of stimulations.

In one embodiment, an implantable device for modulating demyelination and/or remyelination includes an electrode for electrically stimulating the vagus nerve. The electrode can be, for example, a cuff electrode. The electrode can be connected (directly or via a connector) to a controller and signal generator. The signal generator can be configured to provide an electrical signal to the electrode(s). For example, the electrical signal can be an electrical waveform having a frequency of about 0.1 Hz to about 1 KHz (e.g., about 10 Hz), where the pulses applied have a pulse width of about 50 to about 500 usee (e.g., a 200 usee pulse). The signal generator can be a battery (and/or inductively) powered, and the electrical signal can be amplitude and/or voltage controlled. For example, in some embodiments, the device or system can be configured to apply a current that is about 0.05 mA to about 25 mA (e.g., approximately 0.5 mA, 1 mA, 2 mA, 3 mA, etc.). The electrical signal can be sinusoidal, square, random, or the like, and can be charge balanced. In general, the controller (which can be embodied in a microcontroller such as a programed ASIC), can regulate turning on and off the stimulation. For example, stimulation can be applied for a time of about 0.1 sec to about 10 minutes (e.g., about 0.1 sec to about 5 minutes, about 0.1 sec to about 2 minutes, about 1 minute, etc.) at the desired time (e.g., upon successful completion of a motor learning task).

In some embodiments, a VNS implant can be configured to receive control information from a communications device. The communications device can allow modification of the stimulation parameters. In certain embodiments, parameters can include, but are not limited to off-time, on-time, waveform characteristics, duration or other parameter. The communications device can be worn, such as a collar around the neck, or handheld or other suitable attachment.

In other embodiments, methods and systems for modulating demyelination and/or remyelination as described herein can be used in conjunction with one or more pharmacological interventions, and particularly interventions that treat diseases or conditions or injury associated with demyelination, neurodegeneration or neuroinflammation. For example, it can be beneficial to treat a subject receiving stimulation of the vagus nerve to modulate demyelination and/or remyelination by also providing agent such as intravenous corticosteroids (e.g., methylprednisolone), oral corticosteroids, interferons beta-1a and beta-1b, monoclonal antibodies (e.g., natalizumab, alemtuzumab, daclizumab and ocrelizumab), and immunomodulators (e.g., glatiramer acetate, mitoxantrone, fingolimod, teriflunomide, and dimethyl fumarate).

In other embodiments, a motor learning task contemplated herein can include any motor learning task able to induce a learned motor response. In some embodiments, a motor learning task can include simple and precise movements such as a subject walking, raising an arm, juggling a ball, bouncing a ball, or other task.

In some embodiments, a motor skill is a function that involves specific movements of the body's muscles to perform a certain task. These tasks could include walking, running, or riding a bike or even simply moving an arm or leg in a specific position. In order to perform this skill, the bodies nervous system, muscles, and brain has to all work together. In certain embodiments, a goal of motor skills of use herein is to optimize the ability to perform the skill in order to induce an effect on a subject having a condition disclosed herein. Performance as contemplated herein is an act of executing a motor skill or task. Continuous practice of a specific motor skill can result in improved performance or improved ease of performance, which leads to motor learning. Motor learning is a relatively permanent change in the ability to perform a skill as a result of continuous practice or experience.

Motor skills are movements and actions of the muscles. There are two major groups of motor skills, gross and fine motor skills. Gross motor skills require the use of large muscle groups in our legs, torso, and arms to perform tasks such as: walking, balancing, and crawling. The skill required is not extensive and therefore are usually associated with continuous tasks. Gross motor skills can be used repeatedly without putting much thought or effort into them. Gross motor skills can be further divided into two subgroups: Locomotor skills, such as running, jumping, sliding, and swimming; and object-control skills such as throwing, catching, dribbling, and kicking. Fine motor skills require the use of smaller muscle groups to perform smaller movements. These muscles include those found in wrists, hands, fingers, feet and in toes or in the case of other mammals in their hooves or paws, etc. These tasks are precise in nature for example, playing the piano, tying shoelaces, combing hair, brushing teeth, shaving or other fine motor skill. Some fine motor skills may be susceptible to retention loss if not in use, these skills can be lost if not used frequently. Fine motor skills need to continuously be used. Discrete tasks such as switch gears in an automobile, grasping an object, or striking a match, usually require more fine motor skill than gross motor skills. In certain embodiments, gross and/or fine motor skills are included in learned skills contemplated herein alone or in combination with VNS and/or pharmaceutical treatments for a demyelinating condition or disease.

Embodiments of the instant disclosure include kits for applying the devices and methods disclosed herein to a subject having a demyelination condition or disease. In certain embodiments, a kit can include a VNS stimulating device and instructions for a learned motor skill and/or an object of use for a learned motor skill such as a ball or a block or other object. In some embodiments, instructions are provided in the kits for using the device and/or applying the motor skill to the subject. In other embodiments, a kit can further include devices and screening agents for measuring remyelination in a subject and at least one container.

### EXAMPLES

The following examples are included to illustrate certain embodiments and are not considered limiting to the instant disclosure. It should be appreciated by those of skill in the art that the techniques disclosed in the examples which follow represent techniques discovered to function in the practice of the claimed methods, compositions, and apparatus. However, those of skill in the art should, in light of the present disclosure, appreciate that changes can be made in some embodiments and examples which are disclosed and still obtain a like or similar result without departing from the spirit and scope of the invention.

### Example 1

### Motor Cortex Imaging in Transgenic Mice

This example covers protocols for generating mouse neural imaging data, as well as types of mice used for such experiments. All animal experiments were conducted in accordance with protocols approved by the Animal Care and Use Committee at the University of Colorado Anschutz Medical Campus. Male and female mice used in these experiments were kept on 14 hour light/10 hour dark schedules with ad libitum access to food and water, aside from training-related food restriction (see Forelimb Reach Training). All mice were randomly assigned to conditions and were precisely age-matched (±5 days) across experimental groups. Mice expressing eural/glial antigen 2 coupled to membrane anchored EGFP (NG2-mEGFP, Jackson stock #022735) and congenic C57BL/6N MOBP-EGFP (MGI:4847238) lines were used for two-photon imaging. Wild-type C57\B6N Charles River wild-type mice were used in electrophysiological experiments.

Two-photon microscopy: Cranial windows were prepared as previously described¹⁹. Six- to eight-week-old mice were anesthetized with isoflurane inhalation (induction, 5%; maintenance, 1.5-2.0%, mixed with 0.5 L/min O2) and kept at 37° C. body temperature with a thermostat-controlled heating plate. After removal of the skin over the right cerebral hemisphere, the skull was cleaned and a 2×2 mm region of skull centered over the forelimb region of primary motor cortex (0 to 2 mm anterior to bregma and 0.5 to 2.5 mm lateral) was removed using a high-speed dental drill. A piece of cover glass (VWR, No. 1) was then placed in the craniotomy and sealed with Vetbond (3M) and subsequently dental cement (C&B Metabond). A 5 mg/kg dose of Carprofen was administered subcutaneously prior to awakening and for three additional days for analgesia. For head stabilization, a custom metal plate with a central hole was attached to the skull. In vivo imaging sessions began 2-3 weeks post-surgery and took place 2-3 times per week. During imaging sessions, mice were anesthetized with isoflurane and immobilized by attaching the head plate to a custom stage. For MOBP-EGFP experiments, images were collected using a Zeiss LSM 7MP microscope equipped with a BiG GaAsP detector using a mode-locked Ti:sapphire laser (Coherent Ultra) tuned to 920 nm. NG2-mEGFP mice were imaged using a Bruker Ultima Investigator microscope equipped with Hamamatsu GaAsP detectors and a mode-locked Ti:sapphire laser (Coherent Ultra) tuned to 920 nm. The average power at the sample during imaging was 5-30 mW. Vascular and cellular landmarks were used to identify the same cortical area over longitudinal imaging sessions. MOBP-EGFP image stacks were acquired with a Zeiss W “Plan-Apochromat” 20×/1.0 NA water immersion objective giving a volume of 425 um×425 um×336 um (1,024×1,024 pixels; corresponding to layers I-III, 0-336 um from the meninges) from the cortical surface. NG2-EGFP image stacks were acquired with a Nikon LWD Plan Fluorite 16×/0.8 NA water objective with a volume of 805 um×805 um×336 um (2,048×2,048 pixels; corresponding to layers I-III, 0-336 um from the meninges).

SCoRe microscopy: Spectral confocal reflectance microscopy (SCoRe) was performed as described in Schain et al. Nat. Med., 2014; 20: 443-449. For the MOBP-EGFP SCoRE/two-photon validation experiments, in vivo image stacks were acquired on an Olympus F1000MPE upright multiphoton microscope (DIVER). Single-photon confocal microscopy was performed using 488, 543, and 633 nm laser lines combined with appropriate emission filters and descanned Olympus detectors. Two-photon microscopy of MOBP-EGFP fluorescence was performed immediately following SCoRe imaging using a mode-locked Insight X3 laser (Spectra-Physics) tuned to 920 nm and non-descanned Olympus detectors. All images were obtained using an Olympus 20×/1.0 NA water immersion objective (XLUMPLFLN20XW). SCoRe image channels were summed, registered to the two-photon data, and then analyzed for SCoRe/two-photon colocalization using FIJI/ImageJ.

### Example 2

### Cuprizone-Mediated Demyelination

This example outlines a method for controlling demyelination in mice with cuprizone. Cortical demyelination was induced in congenic C57\B6N MOBP-EGFP mice using diets containing 0.2% Cuprizone (bis(cyclohexanone)oxaldihydrazone. The cuprizone was stored in a glass desiccator at 4° C. prior to use. Cuprizone was mixed into powdered chow (Harlan) and provided to mice in custom feeders (designed to minimize exposure to moisture) for three weeks on an ad libitum basis. Feeders were refilled every 2-3 days, and fresh cuprizone chow was prepared weekly. Cages were changed weekly to avoid build-up of cuprizone chow in bedding, and to minimize reuptake of cuprizone chow following cessation of diet via coprophagia. A 3-week partial cortical demyelination model (resulting in 88.3±2.9% oligodendrocyte loss in motor cortex) was used to track the same area of interest over time using surviving oligodendrocytes, and to investigate the behavior of surviving oligodendrocytes.

Given that cuprizone was ingested on a voluntary basis, variation in dosage was controlled in several ways. First, a subset of mice (n=19) were weighed before and after the cuprizone diet to ensure no weight loss had occurred. On average, mice gained weight during cuprizone administration, confirming consumption of the drug (Paired student's t-test, t(18)=2.32, p=0.03). Additionally, variation in maximum oligodendrocyte loss (50-100%) and oligodendrocyte loss and gain had a partially homeostatic relationship in that the amount of loss significantly predicted the subsequent amount of oligodendrocyte gain. To control for variation in total oligodendrocyte loss, and its subsequent effects on oligodendrocyte gain, oligodendrocyte gain was measured relative to the severity of loss using the following equation:

$$oligodendrocyte replacement ( \% ) = \frac{New oligodendrocytes}{Maximum oligodendrocyte loss} ⨯ 100.$$

### Example 3

### Forelimb Reach Tests

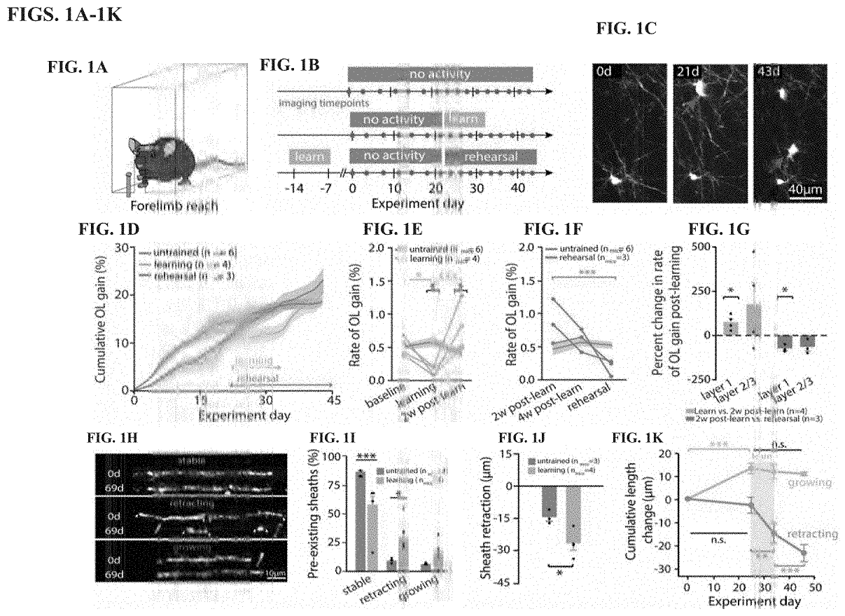

This example covers a forelimb reach test used to measure cognitive ability in mice. A schematic of the testing conditions is provided in FIG. 1A. The mice were deprived of food for 24 hours, and then were weighed and habituated to a training box for 20 minutes prior to forelimb reach training. The training box was fitted with a window providing access to a pellet located on a shelf lcm anterior and lmm lateral to the right-hand side of the window. After one session of initial habituation, training sessions began daily for 20 minutes. Mice learned to reach for the pellet using their left hand. Successes were counted when the mouse successfully grabbed the pellet and transported it inside the box. Errors were qualified in three ways: “Reach error” (the mouse extends its paw out the window but does not grab the pellet), “Grasp error” (the mouse reaches the pellet but does not successfully grasp onto it), and “Retrieval error” (the mouse grasps the pellet but does not succeed in returning it to the box). Mice were kept on a restricted diet throughout training to maintain food motivation but were weighed daily to ensure weight loss did not exceed 10%. For forelimb reach training, mice underwent habituation (average of ˜2 days of exposure) followed by training until seven consecutive days of training with reach attempts were recorded. For the rehearsal of the forelimb reach task, mice performed the reach task during daily 20-minute sessions, 5 days per week over three weeks. To control for any batch or experimenter effects in forelimb reach training results, behavioral performance was only compared for mice trained by the same experimenter within the same batch (e.g., control and experimental mice were only compared if trained at the same time by the same experimenter).

### Example 4

### Immunohistochemistry

This example covers methods for immunohistological analysis in mouse nervous tissue. Mice were anesthetized with an intraperitoneal injection of sodium pentobarbital (100 mg/kg b.w.) and transcardially perfused with 4% paraformaldehyde in 0.1 M phosphate buffer (pH 7.0-7.4). Their brains were postfixed in 4% PFA for 1 h at 4° C., transferred to 30% sucrose solution in PBS (pH 7.4), and stored at 4° C. for at least 24 h. Brains were extracted, frozen in TissuePlus O.C.T, and sectioned coronally or axially (bregma 0 to 2 mm) at 50 um thick. Immunostaining was performed on free-floating sections. Sections were pre-incubated in blocking solution (5% normal donkey serum, 2% bovine g-globulin, 0.3% Triton X-100 in PBS, pH 7.4) for 1-4 h at room temperature, then incubated overnight at 4° C. in primary antibody. Secondary antibody incubation was performed at room temperature for 2 h. Sections were mounted on slides with Vectashield antifade reagent (Vector Laboratories). Images were acquired with a laser-scanning confocal microscope (Zeiss LSM 510).

### Example 5

### Image Processing and Analysis

This example outlines microscopy image processing methods for oligodendrocyte analysis. Image stacks and time-series were analyzed using FIJI/ImageJ. All analysis was performed on unprocessed images except in surviving oligodendrocyte myelin sheath analysis images, which were pre-processed by with a Gaussian blur filter (radius=1 pixels) to denoise. When generating figures, image brightness and contrast levels were adjusted for clarity. For the pseudocolor display of individual myelin sheaths or OPCs, a max projection of the region of interest was generated and was manually segmented and colorized. Longitudinal image stacks were registered using FIJI plugins ‘Correct 3D drift’ or ‘PoorMan3DReg’. When possible, blinding to experimental condition was used in analyzing image stacks from two-photon imaging. To ensure the validity of oligodendrocyte lineage cell tracking, interrater reliability was performed on a subset of images and found a highly significant correlation between raters (R²=0.998, p<0.0001).

For cell tracking, custom FIJI scripts were written to follow oligodendrocytes in four dimensions by defining EGFP+ cell bodies at each timepoint, recording xyz coordinates, and defining cellular behavior (new, dying, proliferating, differentiating, or stable cells). Mature oligodendrocyte and OPC migration, proliferation, death, and differentiation were defined as described below. Differentiation events were recorded as the time point immediately preceding the total loss of NG2-mEGFP fluorescence. OPC and oligodendrocyte gain and loss were quantified cumulatively relative to baseline cell number to account for variation in starting cell number. Rate of oligodendrocyte gain was quantified as the percent change in gain over the amount of time elapsed.

A subset of surviving oligodendrocytes exhibited drastic changes in morphology during remyelination in the form of cell body expansion, sheath addition, and increase in EGFP expression. To ensure that these oligodendrocytes were in fact individual surviving oligodendrocytes and not new oligodendrocytes generated in a similar location and myelinated at similar axonal locations, several criteria were employed for inclusion into the final dataset. Due to the stringency and conservative nature of these criteria, results are likely to underestimate the capacity of surviving oligodendrocytes to generate new myelin sheaths. The criteria included: (1) change in cell soma centroid of less than 2.5 standard deviations from the mean; (2) percentage of sheath retention less than 10% of those of original sheaths; (3) change cell soma volume of less than 700 μm³; (4) protracted sheath addition of greater than 6 days; semi-automated tracing of new sheath to oligodendrocyte surviving cell body (using Simple Neurite Tracer); and (5) distance between surviving and new oligodendrocyte cell bodies at time point of sheath generation of greater than 50 μm.

The change in centroid position of surviving oligodendrocyte cell bodies was measured from baseline to day of peak remodeling (i.e. the day where the largest number of sheaths were added by a given oligodendrocyte). The 3D location of individual surviving oligodendrocytes was ensured to not change by more than 2.5 standard deviations from the mean displacement of reference objects measured within the same stack. Since myelin sheath loss occurs significantly earlier than cell body loss in oligodendrocyte cell death, and a new oligodendrocyte could be generated in the same location immediately following the death of the original oligodendrocyte, only surviving oligodendrocytes that conserved at least 10% of their original processes were included (mean percent conserved processes=75.6±4.45%).

There was an increase in the frequency of pairs and rows of oligodendrocytes with adjacent cell somas in the aging brain. Since the axial resolution of the two-photon microscope was ˜2.6 μm, it was deemed possible to resolve two directly adjacent oligodendrocytes in the z-direction. To ensure that this was the case, several additional steps were taken to rule out the addition of a new oligodendrocyte generated immediately adjacent in the z-axis. As reduction of cell soma size is a hallmark of the maturation of OPCs into myelinating oligodendrocytes, the cell soma volumes of newly generated oligodendrocytes were measured. Next, the change in cell soma volume was measured for each surviving oligodendrocyte at baseline and at the day of peak remodeling. Assuming a z-distance of 0 μm between two cells, oligodendrocytes that grew more than 700 um³ were excluded, and the volume of the smallest new oligodendrocyte measured to ensure that this growth was not unidirectional (as might be observed with the addition of an adjacent oligodendrocyte cell body), but rather that the oligodendrocyte cell body expanded in multiple directions around the centroid position. Previous studies have indicated that new oligodendrocytes have a limited period for myelin sheath generation, for example a few hours in the developing zebrafish and less than 18 hours in vitro. In line with these previous findings, oligodendrocytes were observed to form all sheaths within 0-3 days of generation in mice (nmice=11; ncells=26). Accordingly, for new oligodendrocytes generated directly adjacent to a surviving oligodendrocyte, it was predicted that all additional myelin sheaths would be added within this three day time frame. Therefore, it was required that surviving oligodendrocytes that added more than 4 sheaths—approximately 10% of the average sheaths generated by an oligodendrocyte—must have added these new sheaths over multiple, non-consecutive imaging time points. Using these criteria, two oligodendrocytes were excluded from the final dataset as they may have represented the addition of a new oligodendrocyte in a similar location. One oligodendrocyte violated both the change in centroid requirement and protracted sheath addition requirement (change in centroid=19.3266 um, and 8 sheaths were added at one time point) and the other violated the terms for protracted sheath addition (over 10 sheaths added within two consecutive timepoints). Three other surviving oligodendrocytes were excluded from new sheath analysis because new oligodendrocytes were generated within 50 um of the surviving oligodendrocyte cell body on the day of surviving oligodendrocyte sheath addition. The surviving oligodendrocyte dataset was analyzed by multiple, blinded raters and, where blinding was not possible (e.g. due to recognizable landmarks in the image stacks), all counts were validated by multiple raters. As an additional measure, only new sheaths with processes that could be traced back to the surviving oligodendrocyte cell body with Simple Neurite Tracer were included in the final dataset. Finally, as myelin debris prevented faithful analysis of processes and sheaths from oligodendrocytes in the timepoint following cuprizone treatment, the time point immediately after the removal of cuprizone (i.e. 0 d) was excluded from analysis.

For myelin sheath analysis of individual oligodendrocytes, in vivo z-stacks were collected from MOBP-EGFP mice using two-photon microscopy. Z-stacks were processed with a 1-pixel Gaussian blur filter to aid in the identification of myelin internodes. Myelin paranodes and nodes of Ranvier were identified as described previously, by increase in fluorescence intensity for paranodes and a decrease to zero in EGFP fluorescence intensity for nodes. Myelin sheaths from surviving oligodendrocytes were traced using Simple Neurite Tracer at day −25 and day 21. In normal learning mice, two additional time points were traced that corresponded to day 0 and day 9 of training. To account for differences in measurement due to tracing, a subset of sheaths were traced five times at a single time point. Traces of the same sheath differed by less than 5.56 μm. Therefore, sheaths were defined as stable if their baseline and final lengths changed less than 5.56 μm. Sheaths that grew more than 5.56 μm were considered growing and those that shrank more than 5.56 μm were considered retracting.

For myelin sheath analysis of surviving oligodendrocytes, surviving oligodendrocytes that resided within a volume of 425×425×100 μm³ from the pial surface were considered in Layer I and cells 100-336 μm² were considered Layer Myelin sheaths of surviving oligodendrocytes were tracked throughout time with the same FIJI scripts used for cell tracking. Only sheaths with visible processes back to the surviving cell body in at least one time point were counted. Sheaths were defined as new, lost, or persisting. Persisting sheaths lasted for the entire imaging time course; new sheaths appeared after day 0; and lost sheaths disappeared before the end of the imaging time course and were not visible for at least 2 consecutive timepoints. The average total sheath count per surviving oligodendrocyte was 30.2±1.32. Assuming a normal range of 45±4 sheaths/oligodendrocyte, average sampling revealed 67-74% of all sheaths.

### Example 6

### Electrophysiology

In this exemplary method, electrophysiology methods usable in mouse tissue were studied. Chronic in vivo recordings were conducted during 20-minute forelimb reach training sessions before, during, and after cuprizone treatment. A single 1.6 mm vertical NeuroNexus recording electrode was chronically inserted into primary motor cortex (300 μm anterior to bregma, 1.5 mm lateral bregma) contralateral to the trained forelimb. Data was recorded using Cheetah acquisition software at 30 kHz (NeuroNexus), and single unit activity was clustered using Spike Sort 3D (Neuralynx). Isolation Distance and L-Ratio was used to quantify cluster quality and noise contamination. Spike data was binned at 10 ms and trial-averaged. Heatmaps report average firing rate during 500 ms time window when the animal was not engaged in reach behavior.

### Example 7

### Effects of Forelimb Reach Training on Oligodendrogenesis and Myelination

This example illustrates that learning can shape the pattern of myelination in the healthy and remyelinating tissue, providing the first demonstration of transient learning-induced suppression in oligodendrocyte generation. Tissue regeneration following injury or disease is a long sought-after goal, particularly in the adult nervous system. Oligodendroglia represent one of the few cell types that retain the capacity to regenerate and repair following damage to the adult CNS. Remyelination of denuded axons can restore neuronal function, promote neuroprotection, and may facilitate functional recovery in CNS diseases characterized by myelin loss.

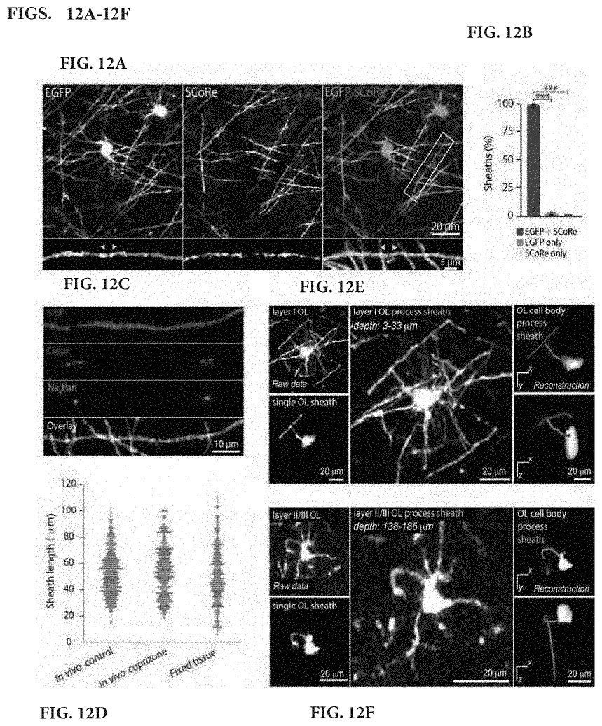

Motor learning can rapidly increase adult oligodendrogenesis, yet the dynamics of activity-dependent myelination remain unclear due to incomplete labeling of differentiating OPC populations and inter-individual variability in cross-sectional approaches. To determine the dynamics of oligodendrocytes, myelin, and OPCs during learning, mice were analyzed with longitudinal two-photon in vivo imaging in the forelimb-region of the motor cortex throughout learning and rehearsal of a skilled, single-pellet contralateral forelimb reach task (depicted in FIG. 1A; Transgenic mice expressing EGFP in all cortical myelinating oligodendrocytes and myelin sheaths (MOBP-EGFP) were interrogated to examine the effects of learning on oligodendrogenesis and preexisting myelin sheath remodeling in healthy mice. Long-term in vivo imaging of layers I-III allowed tracking of approximately 100 oligodendrocytes and their myelin sheaths over the course of 2-3 months per mouse. Immunohistochemistry, in vivo SCoRe imaging, and semi-automated tracing confirmed that EGFP inMOBP-EGFP transgenic mice faithfully reflects the presence and length of myelin sheaths and allows morphological reconstruction of individual oligodendrocytes (FIGS. 12A-12F). Assuming a range of 45±4 sheaths per cortical oligodendrocyte, average sampling revealed between 67% and 74% of individual oligodendrocyte arbors.

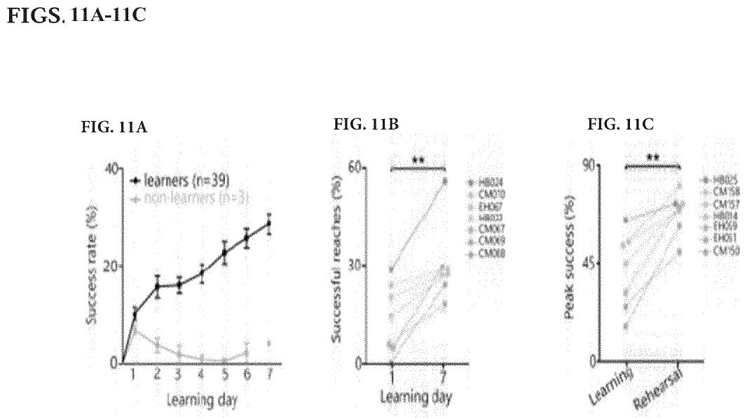

FIG. 1A illustrates a mouse performing a single-pellet contralateral forelimb reach task, as outlined above. FIG. 1B outlines activity and in vivo two-photon imaging schedules for three experimental groups of mice: “untrained” (top), “learning” (middle), and “rehearsal” (bottom). In the figure, blocks labeled “learn” denote once daily 20 minute forelimb reach task training sessions, blocks labeled “rehearsal” denote 20 minute post-training performance of the forelimb reach task, while blocks labeled “no activity” denote periods with no forelimb reach tasks. Imaging timepoints are indicated by dots on each timeline. FIG. 1C provides images showing motor cortex oligodendrogenesis with red arrows indicate new cells. FIG. 1D summarizes cumulative oligodendrogenesis (% increase from baseline; Mean±SEM) by group. FIG. 1E illustrates that learning modulates oligodendrogenesis rate _{(F2,16=15.61,} p=0.0002; grey line±shaded area represents control Mean±SEM; green traces represent individual learning mice). The rate was suppressed during learning relative to baseline (p=0.046; Tukey's HSD), resulting in a decreased rate relative to controls (p=0.016). Rate increases in the two weeks post-learning (p=0.0005; Tukey's HSD), resulting in a higher rate than controls (p=0.05). FIG. 1F displays rates of oligodendrocyte gain, and illustrates that rehearsal modulates oligodendrogenesis rate _{(F2,14=10.33,} p=0.002; grey line±shaded area represents control Mean±SEM; pink traces represent individual rehearsal mice). Rate decreases between two weeks post-learning and rehearsal (p=0.0009; Tukey's HSD), but does not differ between untrained and rehearsal mice. FIG. 1G illustrates percent changes in rates of oligodendrocyte gain following learning periods in multiple layers of the motor cortex, and illustrates that non-zero changes in oligodendrogenesis rate (both the increase two weeks post-learning and decrease during rehearsal) are restricted to layer I of cortex (one sample t-test; p=0.037 and p=0.027, respectively; points represent individual mice). FIG. 1H provides images of stable (top pair of images), retracting (middle pair of images), and growing (bottom pair of images) myelin sheaths. For each image pair, the top and bottom images correspond to the myelin sheaths at zero and 69 days, respectively. FIG. 11 illustrates the proportions of stable, retracting, and growing myelin sheaths in the “untrained” and “learning” groups, and (along with FIG. 1H) illustrates that learning modulates pre-existing sheath stability (%; _{F5,42=69.72,} p<0.0001; points represent means per mouse), and that learning mice have fewer stable sheaths (p<0.0001; Tukey's HSD) and more retracting sheaths (p=0.014; sheaths pseudocolored in h). FIG. 1J provides a comparison of mean myelin sheath retraction lengths for “untrained” (left) and “learning” (right) mice, and illustrates that sheaths retract further in learning vs untrained mice (nmice=4, nsheaths=59 and nmice=3, nsheaths=22, respectively; Student's t-test, t(4.62)=3.32, p=0.02). FIG. 1K provides cumulative myelin sheath length changes in mouse motor cortexes prior to, during, and following 1 week of training in a single-pellet contralateral forelimb reach task. “Growing” sheaths lengthen before learning (Wilcoxon Signed-Rank; p=0.00006) but cease growth after the onset of learning. “Retracting” sheaths are initially stable but retract during (p=0.0047) and after learning (p=0.019). *p<0.05, **p<0.01, ***p<0.001, bars and error bars represent mean±SEM.

FIGS. 11A-11C overviews skill refinement during learning and rehearsal of a forelimb reach task induce skill refinement. FIG. 11A summarizes improvement in single-pellet contralateral forelimb reach task success for learner and non-learner mice. A large majority (93%) of mice successfully learn to perform the forelimb reach task. “Learners” (black) gradually improve their reaching performance over the seven days of training, whereas “non-learners” (grey) show a progressive decrease in success rate and eventually stop making reach attempts around day 4. The lone point in the “non-learner” group at day 7 was likely due to only one mouse making attempts on the last day of training. The other two mice had stopped trying. FIG. 11B provides successful reaches in a single-pellet contralateral forelimb reach task on learning days 1 and 7 for multiple mice. Successful reaches (%) significantly increase between learning days 1 and 7 (paired samples t-test; t(6)=4.80, p=0.003) for mice placed in “learning” group. FIG. 11C summarizes peak success rates in a single-pellet contralateral forelimb reach task on learning days 1 and 7 for multiple mice. Peak performance during rehearsal (successful reaches; %) was significantly higher than peak performance during learning (paired samples t-test; t(6)=5.47, p=0.0016) for mice placed in “rehearsal” group. Individual colors and traces reflect performance by individual mice. *p<0.05, **p<0.01, ***p<0.001. Bars and errors represent Mean±SEM.

FIGS. 12A-12F demonstrate that in vivo imaging ofMOBP-EGFP accurately reflects myelin sheath presence, length, and connection to oligodendrocyte cell body. FIG. 12A provides in vivo imaging of motor cortex neurons from mice with myelin-associated oligodendrocytic basic protein (MOBP) coupled to enhanced green fluorescent protein (EGFP), SCoRe imaging, or a combination of SCoRe imaging on MOBP-EGFP transgenic mice. FIG. 12B compares the percentage of myelin sheaths observed with in vivo imaging of motor cortex neurons from MOBP-EGFP mice, SCoRe imaging, and a combination of SCoRe imaging on MOBP-EGFP transgenic mice. Maximum projections of cortical oligodendrocytes showing 98.24±0.92% colocalization of in vivo MOBP-EGFP and SCoRe signal in myelin sheaths, confirming MOBP-EGFP faithfully reflects presence of myelin (ANOVA, nmice=3, F2,6=5596.220, p <0.0001). FIG. 12C provides maximum projection of 4% paraformaldehyde fixed tissue, stained for myelin (blue, MBP), paranodes (Caspr, red), and sodium channels (NavPan, green). Maximum projection of 4% paraformaldehyde fixed tissue, stained for myelin (blue, MBP), paranodes (Caspr, red), and sodium channels (NavPan, green). FIG. 12D summarizes sheath lengths measured using Simple Neurite Tracer in in vivo two-photon images of control and cuprizone-treated MOBP-EGFP mice, and in confocal images of sheaths immunostained for MBP in fixed tissue. No significant difference between sheath lengths was measured using Simple Neurite Tracer in in vivo two-photon images of control and cuprizone-treated MOBP-EGFP mice, and in confocal images of sheaths immunostained for MBP in fixed tissue (nsheaths=306, 297 and 233, respectively; points represent individual sheaths; ANOVA; F2,833=2.53, p>0.08; red points and error bars represent group meansiSEM). FIG. 12E provides images generated with semi-automated tracing (left and middle) and reconstructions of oligodendrocyte myelin sheaths (right) in the first layer of mouse motor cortexes. FIG. 12F provides images generated with semi-automated tracing (left and middle) and reconstructions of oligodendrocyte myelin sheaths (right) in the second and third layers of mouse motor cortexes. These images collectively demonstrate that semi-automated tracing with Simple Neurite Tracer accurately reconstructs oligodendrocyte myelin sheaths and their connecting processes to the cell soma in layer I (e) and layer II/II (f). Top left: Maximum projection of an oligodendrocyte (OL) imaged using in vivo two-photon microscopy, spanning a depth of 3-33 um (e) and 138-186 um (f) in motor cortex. Bottom left: maximum projection of an isolated single sheath and process attached to the oligodendrocyte cell body. Center: maximum projection and pseudo-colored sheath and process (sheath and process pseudo-colored). Right: Three-dimensional (3D) reconstruction of the same oligodendrocyte generated from the raw in vivo imaging data using the Simple Neurite Tracer plugin in FIJI. View of 3D volume in xy plane from below (top) and view of 3D volume through z (bottom). *p<0.05, **p<0.01, ***p<0.001. Bars and errors represent Mean±SEM.

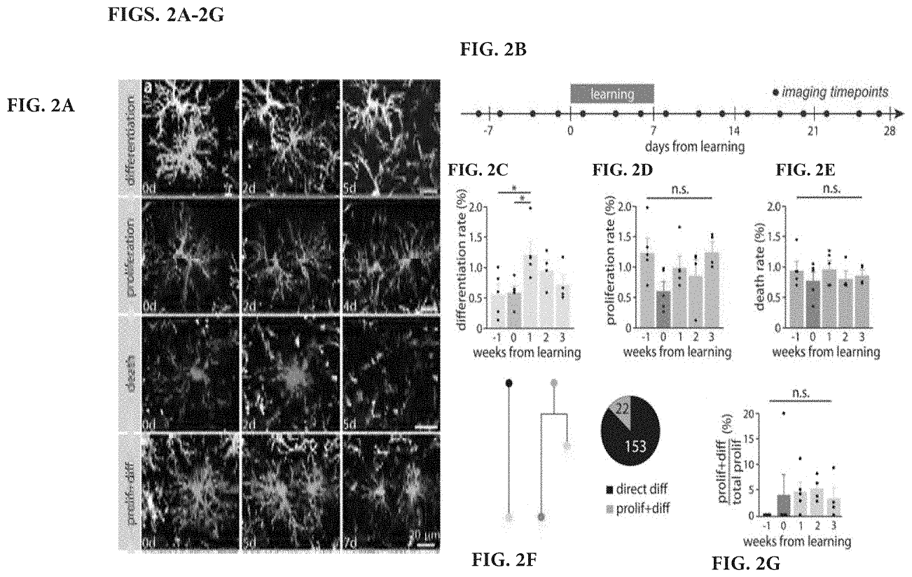

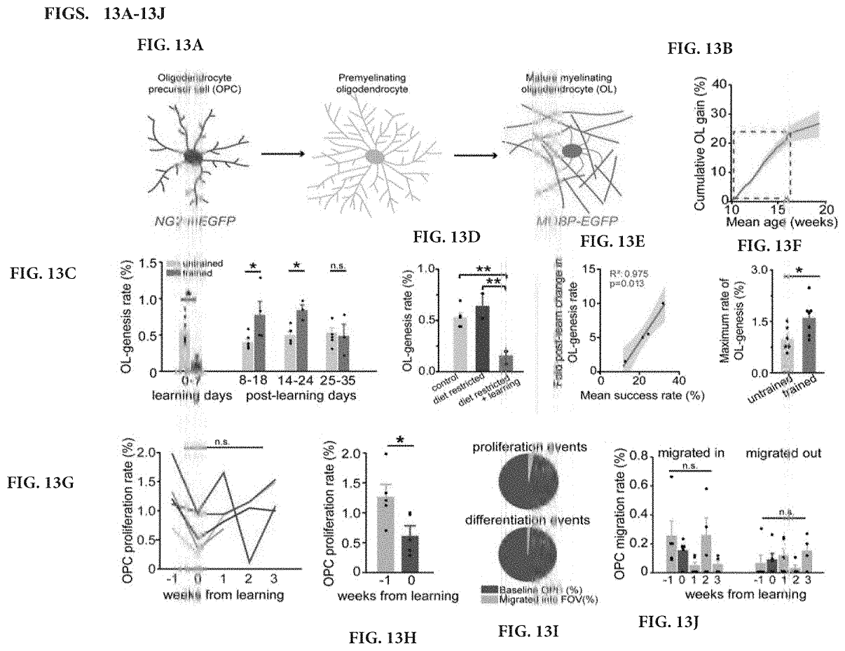

FIGS. 13A-13J outlines oligodendrocyte lineage cell dynamics throughout motor learning. FIG. 13A summarizes genetic lines for oligodenderocyte precursor cells and oligodendrocytes used for in vivo imaging. Genetic lines for in vivo imaging of oligodendrocyte precursor cells (OPCs; NG2-mEGFP) and oligodendrocytes (OLs; MOBP-EGFP). FIG. 13B details motor cortex oligodendrogenesis from age 10-20 weeks across six mice, with the dashed box represents age during standard experimental timeline. Motor cortex oligodendrogenesis from age 10-20 weeks across six mice, showing a plateau ˜17 weeks. Dashed box represents age during standard experimental timeline. FIG. 13C provides oligodendrogenesis rates for the “learning” and “untrained” mice. The rate of oligodendrogenesis was altered in learning vs. untrained mice during learning (Wilcoxon Rank-Sum, p=0.014), days 8-18 post-learning (p=0.038), and days 14-24 post-learning (p=0.024). No differences are observed by days 25-35 post-learning (p>0.9). Points represent mice. FIG. 13D provides oligodendrogenesis rates for diet restricted and diet unrestricted “learning” and “untrained” mice, highlighting that diet restriction can have a pronounced effect on oligodendrogenesis rate (%; ANOVA; F2,8=18.13, p=0.001), as diet-restricted and non-diet-restricted controls have higher rates of oligodendrogenesis than diet-restricted learning mice (Tukey's HSD, p=0.001 and p=0.005, respectively). FIG. 13E compares forelimb reach task success rate with fold change in oligodendrogenesis rate post-treatment. Mean success rate was related to fold change in oligodendrogenesis rate post-learning (R-square=0.98, p=0.01). Line and shaded area represent fit and 95% confidence of fit. FIG. 13F provides maximum oligodendrogenesis rates for the “learning” and “untrained” mice. Trained mice (learning and rehearsal) exhibited increased maximum rates of oligodendrogenesis relative to controls (t(10.61)=−2.49, p=0.03). FIG. 13G show oligodendrocyte precursor cell proliferation rates from FIG. 2D, with different colors representing individual mice. FIG. 13H compare baseline oligodendrocyte precursor cell proliferation rates (left) to oligodendrocyte precursor cell proliferation rates during learning. FIG. 13I provides relative proliferation and differentiation event frequencies All mice showed reduced proliferation rate during learning relative to baseline (t(4)=−3.89, p=0.018; paired student's t-test), but no main effect of time on proliferation rate across the five weeks of experiment—possibly due to high variability post-learning (F4,15=2.341). oligodendrocyte precursor cell that started within the field of view and oligodendrocyte precursor cells which migrated into the field of view during imaging experiments. Only a minority of proliferation and differentiation events occurred in OPCs that had migrated into the field of view throughout the course of the experiment. FIG. 13J provides oligodendrocyte precursor cell migration rates in and out of fields of view during imaging experiments at multiple timepoints relative to learning. Migration into or out of the field of view did not appear to correlate with learning rate. *p<0.05, **p<0.01, ***p<0.001. Bars and errors represent Mean±SEM.

To separate the effects of motor learning from performance, in vivo imaging was performed during initial training phases (“learning” in FIG. 1B; 7 days of 20-minute sessions), or performance of the task one month post-training (“rehearsal” in FIG. 1B; 5 days of 20-minute sessions for 3 weeks). Of all trained mice, 93% were able to learn the task, and both learning and rehearsal of the task resulted in skill refinement (FIGS. 11A-11C). The mice were between 2-3 months old, when oligodendrogenesis is ongoing (FIGS. 13A-13J).

As shown in FIGS. 1D and 1E, learning the reach task appeared to transiently decrease and subsequently increased the rate of oligodendrogenesis in the forelimb motor cortex (FIG. 1d,e). During learning, oligodendrogenesis rate decreased by ˜75% relative to age-matched controls (0.14±0.03% vs. 0.58±0.08%, respectively; rate refers to the % increase in cells over the number of days elapsed). Suppression of oligodendrogenesis was restricted to the training period and was not mediated by the effects of handling (all mice were handled equally) or training-related diet restriction (FIGS. 13A-13J). Immediately following learning, oligodendrogenesis rate increased resulting in an almost two-fold greater rate of oligodendrogenesis relative to untrained controls (0.77±0.19% vs. 0.40±0.04, respectively; FIG. 1E), and remained elevated for 3 weeks (FIGS. 13A-13J). Proficiency in the reach task predicted the magnitude of oligodendrogenesis rate increase following learning (FIGS. 13A-13J). In contrast, rehearsal of the task did not alter oligodendrogenesis rates relative to controls, and the post-learning burst in oligodendrogenesis eventually tapered off (FIG. 1F, FIGS. 13A-13J). Overall, mice that had been trained (both “learning” and “rehearsal”) had higher maximum rates of oligodendrogenesis than untrained mice (FIGS. 13A-13J). Only layer I of forelimb motor cortex demonstrated consistent changes in post-learning oligodendrogenesis rate (FIG. 1G), where motor learning can strengthen horizontal connections between neurons. Oligodendrogenesis rate in layers II/III was variable across mice.

Next, remodeling of pre-existing myelin sheaths throughout learning was considered. Under normal physiological conditions, a small number of myelin sheaths exhibited dynamic length changes (14.7±1.71%; FIGS. 1H, 1I). One week post-learning, the proportion of dynamic pre-existing myelin sheaths increased in learning mice relative to controls (43.46±7.82% vs. 14.74±1.71%). Learning increased both the proportion of sheaths that underwent retraction and the distance these sheaths retracted compared to untrained mice (FIGS. 1I, 1J). Learning also modulated the timing of sheath remodeling; growing sheaths ceased to lengthen at the onset of learning, while learning induced the retraction of previously stable sheaths (FIG. 1K). There was no evidence that new myelin sheaths were generated by pre-existing oligodendrocytes in untrained or learning mice.

To further characterize how motor skill learning modulates the generation of new mature oligodendrocytes, longitudinal in vivo two-photon imaging was used on transgenic mice that express membrane-anchored EGFP in oligodendrocyte precursor cells (OPCs; NG2-mEGFP) to track OPC migration, proliferation, differentiation, and death in forelimb motor cortex over 5 weeks, beginning 1 week prior to forelimb reach training. FIG. 2A provides in vivo images of EGFP-positive OPCs in 10 wk old NG2-mEGFP mice, showing that OPCs that undergo differentiation (yellow; top) retract their filopodia, increase branching, and lose mEGFP fluorescence intensity while surrounding OPC processes infiltrate their domain. Proliferating OPCs (cyan; middle top) undergo cytokinesis and migrate to form independent domains. Dying OPCs (magenta; middle bottom) retract fragmented processes and their cell bodies become enlarged prior to disappearance. A small percentage of OPCs undergo proliferation followed by differentiation (bottom). FIG. 1B provides an experimental timeline with imaging timepoints. FIG. 2C displays differentiation rates for the oligodendrocyte precursor cells during multiple times before, during, and after the forelimb reach task training period, and illustrates that OPC differentiation rate varies by learning week (Mean±SEM; F4,14=4.85, p=0.011). The rate was increased during the first week following forelimb reach training compared to both baseline and learning week (p=0.015 and p=0.021, respectively; Tukey's HSD). FIG. 2D provides proliferation rates for the oligodendrocyte precursor cells during multiple times before, during, and after the forelimb reach task training period. Learning did not appear to affect proliferation rate. FIG. 2E illustrates death rates for the oligodendrocyte precursor cells during multiple times before, during, and after the forelimb reach task training period. Learning did not appear to affect death rate. FIG. 2F compares the proportions of direct (only differentiation) and asymmetric (proliferation and differentiation) differentiation events for the oligodendrocyte precursor cells during 5 weeks of observation. The majority (87.4%) of OPCs underwent direct differentiation (left side of cell fate diagram) as opposed to proliferation followed by differentiation (prolif+diff, right side of cell fate diagram). FIG. 2G outlines ratios of asymmetric and direct differentiation events for the oligodendrocyte precursor cells from 1 week prior to the forelimb reach task training to three weeks after the forelimb reach task training. The proportion of differentiation events that occurred following cell division (prolif+diff) did not differ between baseline and learning or post-learning timepoints. *p<0.05, **p<0.01, ***p<0.001; bars and error bars represent mean±SEM; points represent individual mice.

Similar to oligodendrogenesis, learning induced a two-fold increase in the rate of OPC differentiation in the week following learning (FIG. 2C, 0.59±0.10% during learning vs. 1.23±0.19% post-learning), yet OPC differentiation rate was unaffected during reach training. Neither the rates of proliferation nor death differed significantly across the five weeks (FIGS. 2D-2E). However, 5/5 mice displayed a reduction in proliferation rate (˜50%) during learning relative to baseline (FIGS. 13A-13J).

A majority of adult OPC differentiation events may occur via direct differentiation rather than asymmetric cell division. In line with this hypothesis, it was found that only 10.91±3.77% of differentiating OPCs had previously proliferated during the 5 weeks of observation. The proportion of asymmetric differentiation events was unaffected by motor learning (FIGS. 2A, 2F, 2G). To assess whether the increase in OPC differentiation following motor learning was due to parenchymal OPCs or precursors recruited from nearby brain regions or germinal zones, OPCs that migrated into the imaging volume were actively tracked (FIGS. 13A-13J). Migration into the field was rare, with 4.68±0.96% of the baseline number of OPCs migrating in and 2.73±0.36% migrating out and was not altered by learning. Only 3.57±1.49% of the total proliferation events and 0.70±0.30% of the total differentiation events occurred in cells that migrated into the imaging volume. These data indicate that parenchymal OPCs residing in motor cortex directly differentiated following acquisition of the reach task.

### Example 8

### Effects of Demyelination on Oligodendrocyte Replacement, Myelination, and Functional Deficits in the Motor Cortex

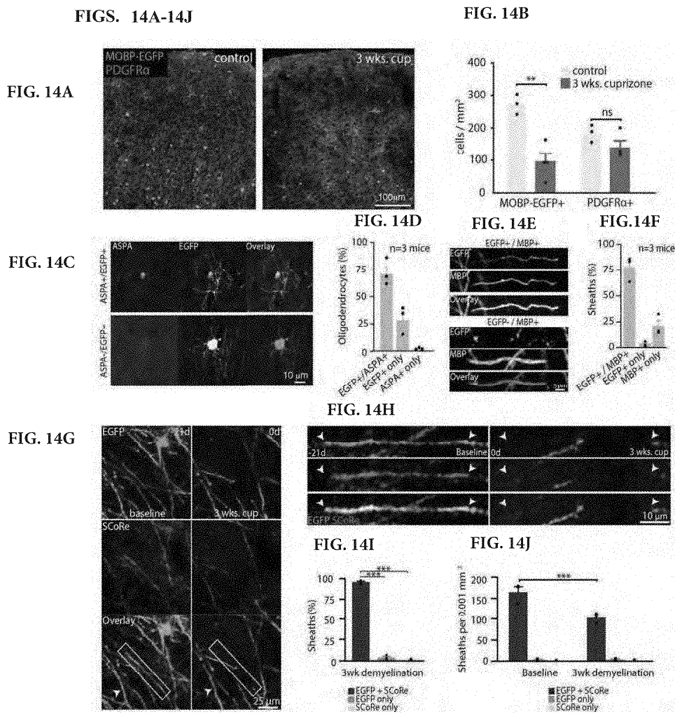

This example covers oligodendrocyte replacement, myelination, and functional deficits in the motor cortex following demyelination. Gray matter lesions in patients with MS contain both dying and newly forming oligodendrocytes, a feature that has complicated the interpretation of remyelination in humans and animal models of MS. To visualize the dynamics of myelin loss and repair, longitudinal two-photon in vivo imaging was used during cuprizone-mediated demyelination (FIG. 3A). 10-week-old congenic MOBP-EGFP mice were fed a 0.2% cuprizone diet for three weeks to induce oligodendrocyte death (˜90% in forelimb motor cortex, FIGS. 3A-3H), and confirmed that in vivo two-photon analysis of MOBP-EGFP mice is a reliable measure of oligodendrocyte and myelin sheath loss with SCoRe and immunohistochemistry (FIGS. 14A-14J). In contrast to the loss of myelin and mature oligodendrocytes, the number of cortical OPCs was unchanged following cuprizone administration relative to age-matched controls (FIGS. 14A-14J, 138.63±19.75 cells/mm² vs. 179.11±14.99 cells/mm², respectively).

FIGS. 3A-3J illustrates that demyelination can result in incomplete oligodendrocyte replacement and functional deficits in motor cortex. FIG. 3A provides a timeline of cuprizone administration and in vivo two-photon imaging. FIG. 3B provides images of oligodendrocytes following cuprizone administration prior to (left) and immediately following (middle) the cuprizone diet outlined in FIG. 3A, as well as for mice on a cuprizone-free diet (right). Surviving cells are shown in grey, dead cells are shown in red, and new cells are shown in blue. FIG. 3C compares lifespans for oligodendrocytes following cuprizone administration, categorized as “surviving” (grey), “lost” (red), and “new” (blue). The shaded area represents the timeframe for cuprizone administration. FIG. 3D provides images of EGFP+ myelin sheaths in mice three weeks prior to cuprizone administration (left), immediately following the completion of the cuprizone regimen (second from left), 5 days following the cuprizone regimen (third from left), and 7 days following cuprizone treatment (right) FIG. 3E provides timelines for myelin loss and cell body loss in mice subjected to cuprizone treatment, and displays a biphasic oligodendrocyte loss profile: initial loss of EGFP+ myelin sheaths and subsequent shrinking of cell body before loss of EGFP signal in an MOBP-EGFP mouse. Myelin loss (nmice=3, ncells=45) occurs earlier than oligodendrocyte soma loss (nmice=3, ncells=47; Student's t-test, t(90)=−5, p<0.0001; box plots represent median and IQR). FIG. 3F details oligodendrocyte gain and loss in multiple mice over three weeks of cuprizone treatment and the three weeks following. Cumulative oligodendrocyte gain and loss relative to baseline (%); traces represent individual mice. FIG. 3G compares cumulative oligodendrocyte loss and gain relative to the baseline measurement, and suggests that cumulative oligodendrocyte loss is tightly related to oligodendrocyte gain (Spearman's ρ=0.922, p<0.0001). FIG. 3H compares changes in proportions of lost and replaced oligodendrocytes during and after cuprizone treatment, showing a delayed inflection point for oligodendrocyte replacement relative to loss (8.71±0.72 vs. 4.51±0.68 days post cuprizone, respectively; nmice=5; t(8)=4.24, p=0.0028; Student's t-test), and decreased asymptote of replacement relative to loss 60.52±3.03% vs. 87.06±3.10%, respectively; t(8)=6.12, p=0.0003; Student's t-test). FIG. 31 provides representative heat maps of neuronal firing rate (FR) in the motor cortex of healthy mice (left, control) versus remyelinating mice (right, cuprizone). FIG. 3J compares neuronal firing rates at multiple timepoints during and after cuprizone-treatments in mice. Neuronal FR was comparable between control and cuprizone mice both prior to and during cuprizone administration, but was elevated in cuprizone mice both in the first and second week following cuprizone cessation (Wilcoxon Rank-Sum; p=0.0063 and p=0.0157, respectively; points represent individual neurons, lines and error bars represent median and IQR). By three weeks post-cuprizone, FR was indistinguishable between cuprizone and control mice. *p<0.05, **p<0.01, ***p<0.001.

FIGS. 14A-14J summarizes loss of myelin and oligodendrocytes during cuprizone treatment. FIG. 14A provides mouse motor cortex images showing MOBP-EGFP and platelet derived growth factor alpha (PDGFRa) prior to (left) and post (right) cuprizone treatment. FIG. 14B provides MOBP-EGFP and PDGFRa levels in mouse motor cortexes prior to (left) and post (right) cuprizone treatment. Mean density of EGFP+and PDGFR in the soma suggest it was a recently born oligodendrocyte in the early stages of the maturation process. FIG. 14C provides mouse motor cortex images with ASPA and EGFP channels. The top row corresponds to ASPA+/EGFP+ cells, while the bottom row corresponds to ASPA−/EGFP+ cells. FIG. 14D summarizes the percentage of oligodendrocytes which are EGFP+/ASPA+, EGFP+, and ASPA+ after three weeks of cuprizone treatment. After three weeks of cuprizone treatment, 70.73±12.78% of oligodendrocytes were EGFP⁺/ASPA⁺, 0.97±0.84% of cells were ASPA⁺/EGFP⁻, while the remainder were EGFP⁺-only (nmice=³, ncells=¹⁸⁵). FIG. 14E provides maximum projections of MBP⁺ myelin sheaths with (top) and without EGFP (bottom) after three weeks of cuprizone. Maximum projection of an MBP⁺ myelin sheath with (top) and without EGFP (bottom) after three weeks of cuprizone. FIG. 14F summarizes the percentage of myelin sheaths which are EGFP+/MBP+, EGFP+, and MBP+ after three weeks of cuprizone treatment. After three weeks of cuprizone, 76.21±7.11% of sheaths were MBP⁺/EGFP⁺, and 20.6±5.79% of sheaths were MBP⁺/EGFP⁻ (nmice=3, nsheaths=351). FIG. 14G provides mouse motor cortex images with EGFP, SCoRe, and overlayed channels prior to (left) and following (right) 3 weeks cuprizone treatment. FIG. 14H provides maximum projections of oligodendrocytes showing colocalization of in vivo MOBP-EGFP and SCoRe imaging for myelin both before cuprizone administration (−21 days) and immediately following its removal (0 days). Note the surviving sheath (white arrow). FIG. 141 summarizes the percentage of myelin sheaths in FIG. 14G resolved with EGFP and SCoRe channels versus EGFP or SCoRe channels only. Following 3 weeks of cuprizone diet, most myelin sheaths were MOBP-EGFP⁺/SCoRe⁺ (95.71±1.16; ANOVA, F2,6=2012.94, p<0.0001). FIG. 14J summarizes the density of myelin sheaths (per 0.01 mm³) resolved with EGFP and SCoRe channels versus EGFP or SCoRe channels only prior to and following 3 weeks cuprizone treatment. Cuprizone administration was shown to modulate sheath density (F2,10=14.43, p=0.001). Cuprizone-fed mice have a reduced density of MOBP-EGFP⁺/SCoRe⁺ positive sheaths relative to controls (p=0.0001), but no difference in GFP-only or SCoRe-only sheaths. *p<0.05, **p<0.01, ***p<0.001. Bars and errors represent Mean±SEM.

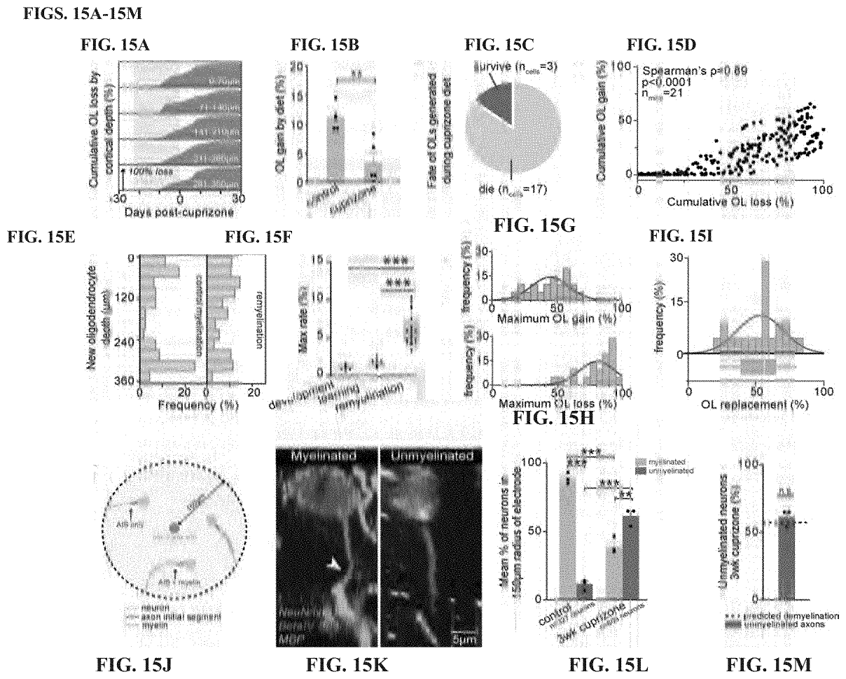

FIGS. 15A-15M outlines dynamics of oligodendrocyte generation and loss during cuprizone treatment. FIG. 15A summarizes oligodendrocyte loss in mouse motor cortex tissue as a function of cortical depth and days post-cuprizone treatment, with the gray shaded region indicating the period of cuprizone treatment. Oligodendrocyte loss occurred evenly across cortical depths. Shaded area represents cuprizone diet. FIG. 15B summarizes oligodendrocyte gain as a function of diet (cuprizone (left) versus non cuprizone (right)). Oligodendrogenesis was suppressed during cuprizone diet (n=5 mice per group; t(6.54)=4.10, p=0.005; Student's t-test). FIG. 15C compares survival and death prevalency among oligodendrocytes generated during 3-week cuprizone diets. 85% of oligodendrocytes generated during cuprizone diet die within three weeks. FIG. 15D compares cumulative oligodendrocyte gain and loss during a cuprizone treatment regimen. Oligodendrocyte loss predicts gain (Spearman's maximum projection) were co-labelled with MBP (myelin; cyan), beta-IV spectrin (axon initial segment; purple) and NeuN/NFH (neuron cell soma/distal axon; green; top), whereas unmyelinated axons did not co-localize with MBP (bottom). FIG. 15E provides relative cortical depth frequencies for new oligodendrocytes generated during a cuprizone treatment regimen. FIG. 15F summarizes maximum rates of oligodendrocyte growth during development, learning, and remyelination phases in mouse motor cortexes. FIGS. 15G-15H provides relative frequencies for maximum oligodendrocyte gain and loss in mouse motor cortexes over a cuprizone treatment regimen. FIG. 15I provides relative frequencies for oligodendrocyte replacement in mouse motor cortexes over a cuprizone treatment regimen. FIG. 15J provides a diagram depicting axons with various degrees of myelination. FIG. 15K provides images of myelinated (left) and unmyelinated (right) axons, with the axons shown in green, the axon initial segments shown in purple, and myelin shown in blue. FIG. 15L provides a comparison of myelinated and unmyelinated neurons within 150 micron radius of an electrode in cuprizone treated and non-treated samples. Cuprizone administration altered pen-probe axonal myelination (Two-way ANOVA; F3,8 =110.51, p<0.0001). Control mice had more myelinated versus unmyelinated axons (Tukey's HSD, p<0.0001). At the cessation of cuprizone, cuprizone-fed mice had fewer myelinated (p<0.0001) and more unmyelinated axons than healthy controls (p<0.0001), and more unmyelinated than myelinated axons (p=0.004, note that myelin may be present elsewhere on the axon). FIG. 15M illustrates predicted versus measured percentages of unmyelinated neurons following three weeks of cuprizone treatment. The proportion of unmyelinated neurons observed via IHC does not differ from the proportion of myelin loss predicted by sigmoidal demyelination characterized in FIG. 3E-3H (one-sample t-test, t(2)=1.10, p>0.3). *p<0.05, **p<0.01, ***p<0.001. Bars and errors represent Mean±SEM.

Oligodendrocyte loss occurred evenly across cortical depths (FIGS. 15A-15M), leaving a small number of oligodendrocytes and myelin intact (12.94±3.10%, “surviving” oligodendrocytes; FIGS. 3B, 3C). Cuprizone treatment suppressed oligodendrogenesis, and 85% of the few cells generated during cuprizone administration died within 3 weeks (FIGS. 15A-15M). Oligodendrocyte death followed a biphasic model of myelin loss (1.53±1.27 days before cuprizone cessation) followed by cell body loss (7.26±1.22 days post-cuprizone; FIGS. 3D, 3E), similar to previous descriptions of demyelination occurring via a “dying-back” process. Oligodendrocyte loss plateaued approximately fifteen days following the cessation of cuprizone administration (FIG. 3F). Cuprizone diet removal induced a robust oligodendrogenesis response that was proportional to the extent of oligodendrocyte loss and that plateaued at approximately three weeks (FIGS. 3F, 3G; FIGS. 15A-15M). The cortical distribution of newly generated oligodendrocytes was comparable in remyelinating and healthy conditions. Maximum oligodendrogenesis rates during remyelination were six times greater than in healthy untrained mice (5.95±0.38% vs. 0.99±0.69%) and almost four times greater than in healthy trained mice (5.95±0.38% vs. 1.60±0.64%; FIGS. 15A-15M).

To further characterize the oligodendrogenesis response, mice were tracked up to 60 days after cuprizone cessation. Due to the oral uptake of cuprizone, there was inter-mouse variation in the extent of demyelination, and consequently, remyelination (FIGS. 15A-15M). Oligodendrocytes generated during remyelination were therefore quantified as a proportion of total oligodendrocyte loss (“oligodendrocyte replacement”). Oligodendrocyte replacement after cuprizone cessation followed a sigmoidal pattern, and was quantified using three-parameter (3P) logistic equations, which captured the inflection point (when oligodendrogenesis switches from accelerating to decelerating) and the asymptote of the curve (the plateau of oligodendrocyte replacement). Oligodendrocyte replacement was delayed relative to loss by approximately four days and plateaued significantly lower than oligodendrocyte loss (FIG. 3H). Mice only replaced on average 60.52±3.03% of lost oligodendrocytes in the seven weeks post-cuprizone; the remyelination response failed to restore baseline oligodendrocyte numbers.

To determine the effects of oligodendrocyte loss and incomplete replacement on neuronal function in forelimb motor cortex, chronic weekly in vivo extracellular recordings were performed in both cuprizone-demyelinated and age-matched control mice. In vivo multi-site electrodes record extracellular potentials from neurons within approximately a 150 micron radius of the recording electrode. Histology confirmed that the proportion of proximally-myelinated neurons in this sampling radius decreased by over 50% at the cessation of cuprizone administration relative to controls (FIGS. 15A-15M). Neuronal firing rates did not differ between groups prior to or during cuprizone administration. However, median neuronal firing rates were increased in demyelinated mice versus controls by ˜70% in the first week and 40% in the second week post-cuprizone (11.90 vs. 6.92 Hz and 10.68 vs. 7.69 Hz, respectively; FIGS. 3I-3J), indicating they were hyperexcitable in a manner that temporally correlates with maximum oligodendrocyte loss. By three and four weeks post-cuprizone—when remyelination plateaued—neuronal firing rates in cuprizone-demyelinated mice were indistinguishable from age-matched controls. Taken together, these results demonstrate that cuprizone-mediated demyelination induces aberrant neuronal function in the forelimb region of motor cortex that recovers synchronously with remyelination.

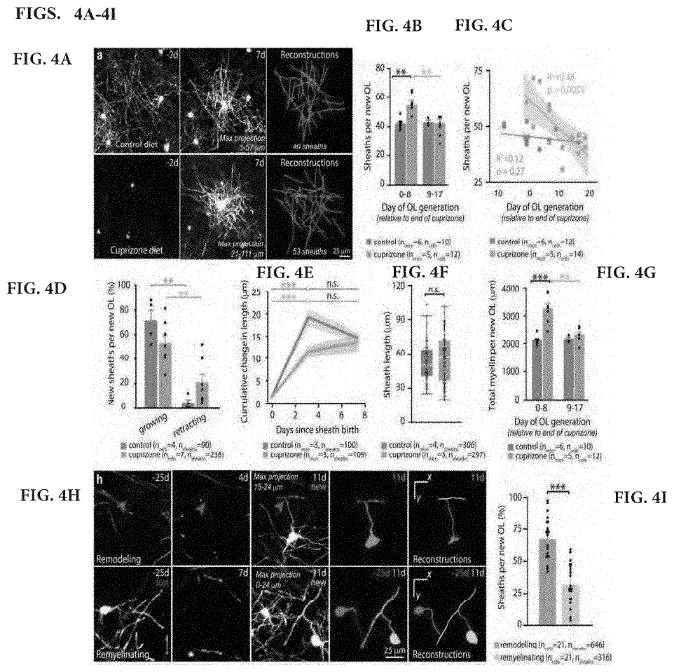

Given that remyelination failed to completely restore baseline oligodendrocyte number but seemed to restore neuronal function, the number, length, and location of sheaths generated by new oligodendrocytes during remyelination were examined. FIGS. 4A-4I suggests that myelin sheath number on new oligodendrocytes is regulated during remyelination. FIG. 4A provides motor cortex images for mice raised on cuprizone-free diets (top, “control”) and on the 0.2% cuprizone diet. FIG. 4B provides the number of myelin sheaths per new oligodendrocyte in the “control” (grey) and “cuprizone” (purple) mice 8 and 17 days after cuprizone administration. Remyelination modulates sheath number _{(F1,19=8.03,} p=0.0105). Oligodendrocytes generated in the first week of remyelination (top, a) generate more sheaths than age-matched control oligodendrocytes (bottom, a; p=0.010, Tukey's HSD) or than oligodendrocytes formed after week 1 of remyelination (p=0.0023). FOV shown 2 days before oligodendrocyte birth and 7 days post-birth. Points represent individual oligodendrocytes. FIG. 4C illustrates the number of myelin sheaths per new oligodendrocyte for “control” (grey) and “cuprizone” (purple) mice at different timepoints relative to cuprizone administration, with day 0 corresponding to the first day following cuprizone treatment. Day of oligodendrocyte generation relative to end of cuprizone predicts sheath number in demyelinated mice (R²=0.48, _{F1,12=11.16,} p=0.006; shaded area represents 95% confidence of fit; points represent oligodendrocytes). FIG. 4D compares the number of new sheaths between growing and retracting new oligodendrocytes for “control” (grey) and “cuprizone” (purple) mice. In the first three days post-generation, sheaths from new oligodendrocytes grow more often than they retract (F3,18=15.34, p<0.0001) in both control (p=0.0001, _{nmice=4)} and cuprizone-treated mice (p=0.0096, nmice=6, Tukey's HSD). Points represent individual oligodendrocytes. FIG. 4E provides cumulative changes in length (in p.m) following sheath birth in “control” (grey) and “cuprizone” (purple) mice. New sheaths change in length in the week following their generation (control: F3,302=47.94, p<0.0001, cuprizone: F3,293=29,71, p<0.0001; lines and shaded area represent mean±SEM). Sheaths in both control and cuprizone treatment stabilize their length within 3 days of sheath birth (d0 vs. d3, p<0.0001 in both treatments; Tukey's HSD). FIG. 4F displays average myelin sheath lengths (in p.m) in new oligodendrocytes in “control” (grey) and “cuprizone” (purple) mice. Sheath length does not differ in control and cuprizone-treated mice 3 days after sheath generation (boxplots represent median and IQR; points represent sheaths). FIG. 4G illustrates total myelin per new oligodendrocytes in control (grey) and cuprizone fed (purple) mice. Remyelination shapes predicted total myelin ([mean sheath length]×[# of sheaths/OL]) generated by a new oligodendrocyte (F1,19=8.93,p=0.0077). It was higher in week 1 of remyelination than age-matched control oligodendrocytes (p<0.0001) or than oligodendrocytes generated after week 1 of remyelination (p=0.0016; Tukey's HSD). FIG. 4H provides mouse motor cortex images of newborn oligodendrocytes in previously unmyelinated (top, “Remodeling”) and myelinated (bottom, “Remyelinating”) areas at different timepoints relative to cuprizone treatment, showing that new oligodendrocytes can place sheaths in previously unmyelinated areas (top, “Remodeling”) or previously myelinated areas (bottom, “Remyelinating”). Pink arrows point to location of junction between new sheath and new OL process. Relevant sheaths pseudo-colored. FIG. 4I provides the number of myelin sheaths per new oligodendrocyte in “Remodeling” and “Remyelinating” portions of mouse motor cortexes. New oligodendrocytes engage in remodeling more often than remyelinating (t(20)=−5.08, p<0.0001, _{nmice=5;} Paired student's t-test). *p<0.05, **p<0.01, ***p<0.001; bars and error bars represent mean±SEM.

In the first week of remyelination, new oligodendrocytes formed more myelin sheaths than oligodendrocytes generated in the second week of remyelination or in control mice (54.4±3.25 vs. 39.4±1.72 and 42.28±1.30 total sheaths, respectively; FIGS. 4A-4C). In healthy mice and during remyelination, sheaths from new oligodendrocytes stabilized to similar lengths within three days after generation (FIGS. 4D-4F). Therefore, the increased sheath number on new oligodendrocytes in the week following demyelination resulted in a larger total amount of myelin per oligodendrocyte (FIG. 4G). In addition, it was found that myelin sheaths of newly generated oligodendrocytes were more often placed in previously unmyelinated areas (“remodeling”; 67.7±3.56% of sheaths) rather than in denuded areas (“remyelinating”; 32.0±3.47% of sheaths), generating a novel pattern of myelination following demyelinating injury (FIGS. 4H-4I). These findings indicate that the myelinating capacity of individual oligodendrocytes was increased during early remyelination, and that remyelination by new oligodendrocytes alters the pattern of cortical myelin.

### Example 9

Motor Learning Modulates Oligodendrogenesis after Demyelination in a Timing-Dependent Manner

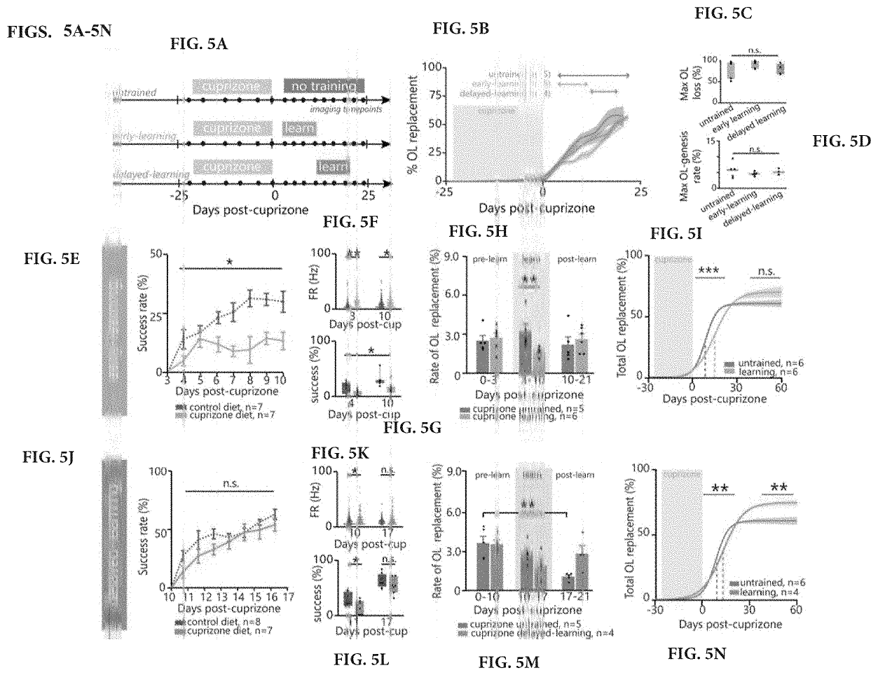

This example details time dependencies of learning-mediated oligodendrogenesis following demyelination. Since it was found that motor learning increased both OPC differentiation and oligodendrogenesis in healthy mice (FIGS. 1A-1K, FIGS. 2A-2G), it was next examined whether motor learning could enhance oligodendrocyte replacement in demyelinated mice. Mice were allotted to one of three experimental groups: “no activity,” “early-learning” (starting 3 days post-cuprizone), and “delayed-learning” (starting 10 days post-cuprizone; FIG. 5A). Behavioral intervention had no effect on the severity of demyelination (FIG. 5C) nor the maximum rate of oligodendrogenesis during remyelination (FIG. 5D).

FIGS. 5A-5N illustrates that learning can modulate oligodendrogenesis after demyelination in a timing-dependent manner. FIG. 5A outlines learning, cuprizone treatment, and imaging schedules for three experimental groups of mice: “untrained” (top), “early-learning” (middle), and “delayed-learning” (bottom). In the figure, blocks labeled “learn” denote once daily 20 minute forelimb reach task training sessions, blocks labeled “cuprizone” periods in which mice were provided 0.2% cuprizone diets, while blocks labeled “no training” indicate mice which were not provided forelimb reach task training sessions. FIG. 5B provides oligodendrocyte replacement for the “untrained,” “early-learning,” and “delayed-learning” mice, showing cumulative OL replacement (%; lines and shaded areas represent mean±SEM) across post-cuprizone behavioral interventions. FIG. 5C provides max oligodendrocyte loss during cuprizone treatment for the “untrained,” “early-learning,” and “delayed-learning” mice. FIG. 5D provides max oligodendrocyte genesis rates following oligodendrocyte loss during cuprizone treatment for the “untrained,” “early-learning,” and “delayed-learning” mice. Neither maximum OL loss nor maximum rate of oligodendrogenesis differ between behavioral interventions (boxplots represent median and IQR). FIG. 5E provides forelimb reach task success rates for mice administered either cuprizone or control diets on the early-learning regimen. Demyelination modulates early-learning success rate _{(F6,78=3.00,} p=0.011, points represent mean±SEM). Success rate improves from first to last day of reaching for control (p=0.005; Tukey's HSD), but not cuprizone-treated mice. FIG. 5F provides neuron firing rates for mice administered either cuprizone or control diets on the early-learning regimen. Both 3 and 10 days post-cuprizone, demyelinated mice have increased neuronal FR relative to controls (Wilcoxon Rank-Sum, p=0.006 and p=0.016, respectively; points represent neurons). FIG. 5G summarizes forelimb reach task success rates for mice administered either cuprizone or control diets on the early-learning regimen. Both 4d post-cuprizone and 10d post-cuprizone, demyelinated mice have decreased success rates relative to controls _{(F1,13=9.09,} p=0.01; points represent mice). FIG. 5H compares rates of oligodendrocyte replacement prior to learning, during learning, and post-learning for mice administered either cuprizone or control diets on the early-learning regimen. OL replacement rate was suppressed during early-learning relative to untrained demyelinated mice (Wilcoxon Rank-Sum, p=0.0043). FIG. 5I compares cumulative oligodendrocyte replacement in mice administered either cuprizone or control diets on the early-learning regimen, with the gray shaded area indicating the period of cuprizone-treatment. Delayed inflection point of OL-replacement in early-learning vs. untrained demyelinated mice (Student's t-test; t(10)=5.77, p=0.0002), colored line/shaded area represents asymptote±SEM. FIG. 5J provides forelimb reach task success rates for mice administered either cuprizone or control diets on the delayed-learning regimen. No effect of cuprizone treatment on overall delayed-learning performance. FIG. 5K provides neuron firing rates for mice administered either cuprizone or control diets on the delayed-learning regimen. 10 days post-cuprizone, but not 17 days post-cuprizone, demyelinated mice showed increased neuronal FR relative to controls (Wilcoxon Rank-Sum, p=0.016). FIG. 5L summarizes forelimb reach task success rates for mice administered either cuprizone or control diets on the delayed-learning regimen. 11 days post-cuprizone, but not 17 days post-cuprizone, demyelinated mice have impaired reaching performance relative to controls (Student's t-test; t(12.28=−2.39, p=0.033). FIG. 5M compares rates of oligodendrocyte replacement prior to learning, during learning, and post-learning for mice administered either cuprizone or control diets on the delayted-learning regimen, showing that delayed-learning modulates OL replacement rate _{(F2,14=4.61,} p=0.029). Rate decreases in untrained, but not delayed-learning, mice by 21 days post-cuprizone (p=0.008; Tukey's HSD). FIG. 5N, Delayed inflection point (Student's t-test; t(8)=4.33, p=0.0025) and increased asymptote of OL-replacement (t(8)=3.35, p=0.01) in delayed-learning vs. untrained mice. *p<0.05, **p<0.01, ***p<0.001, bars and error bars represent mean±SEM.

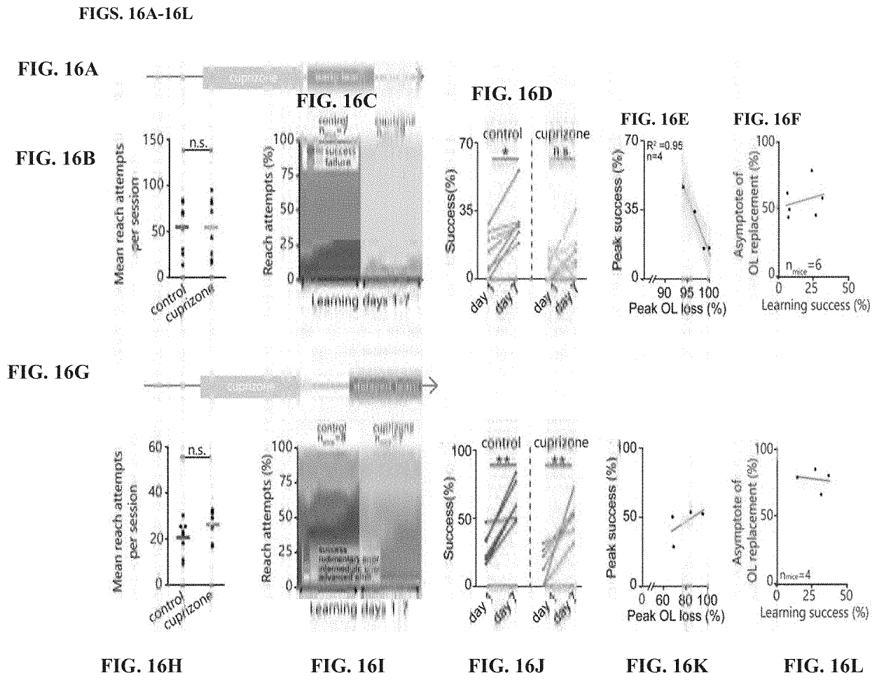

FIGS. 16A-16L illustrates demyelination induced deficits during early, but not delayed, motor learning. FIG. 16A provides a timeline for “early-learning” intervention (3 days post-cuprizone). FIG. 16B provides mean reach attempts per learning session in forelimb reach tasks for cuprizone treated and untreated mice provided the learning regimen. No difference in mean reach attempts per session were observed for early-learning between control and cuprizone-treated mice (Student's t-test, t(12.95)=0.05, p>0.9; colored lines represent group means). FIG. 16C summarizes successful and failed reach attempts in forelimb reach tasks over 7 learning days for cuprizone treated and untreated mice for the learning regimen Area plot of reach attempt outcome (success vs. failure) across forelimb reach learning days in both control and cuprizone-treated mice. FIG. 16D summarizes forelimb reach task success rates for cuprizone treated and untreated mice provided the learning group of mice. Control mice showed improved success rates day 7 of training relative to day 1 (Paired Student's T-test; t(6)=4.7, p=0.003), while cuprizone-treated mice did not (t(7)=1.96, p=0.09). FIG. 16E provides peak forelimb reach task success rates as a function of peak oligodendrocyte loss in mice provided the learning group. Maximum oligodendrocyte loss was related to peak performance during training (R²=0.95, p=0.02; line and shaded area represent line of fit and 95% confidence). FIG. 16F summarizes asymptotes of oligodendrocyte replacement as a function of forelimb reach task learning success for six mice provided the learning regimen. No relationship between mean learning success rate (%) and asymptote of oligodendrocyte replacement was observed in early learning mice. FIG. 16G summarizes a timeline for “delayed-learning” intervention (10 days post-cuprizone) FIG. 16H provides mean reach attempts per learning session in forelimb reach tasks for cuprizone treated and untreated mice provided the learning regimen. No difference in mean reach attempts per session during delayed-learning were observed between control and cuprizone-treated mice (Student's t-test, t(12.95)=1.54, p>0.1; coloured lines represent group means). FIG. 16I provides area plot of reach attempt outcome (success, rudimentary error, intermediate error, advanced error; see Supplementary Video 1) across delayed-learning days in both control and cuprizone-treated mice. FIG. 16J, both control and cuprizone-treated mice improved their reaching success between days 1 and 7 of delayed-learning (Paired student's t-test, p=0.0005 and p=0.004, respectively). FIG. 16K provides peak forelimb reach task success rates as a function of peak oligodendrocyte loss in mice provided the learning regimen. No relationship between maximum oligodendrocyte loss and reaching performance during delayed learning was observed. FIG. 16L summarizes asymptotes of oligodendrocyte replacement as a function of forelimb reach task learning success for six mice provided the learning regimen. No relationship between delayed learning success rate and asymptote of oligodendrocyte replacement post-cuprizone was observed. *p<0.05, **p<0.01, ***p<0.001. Points represent individual mice.

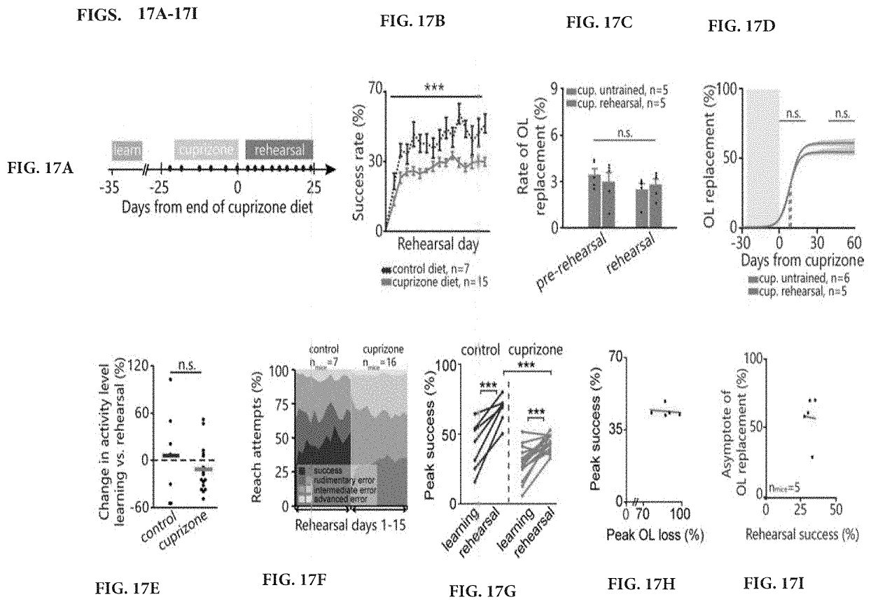

FIGS. 17A-17I demonstrates that motor skill rehearsal does not modulate remyelination. FIG. 17A provides a timeline for mouse reach task rehearsal following a cuprizone diet. FIG. 17B summarizes forelimb reach task success rates for mice raised on cuprizone-containing and cuprizone-free diets, and highlighting the effect of drug treatment on reaching success during rehearsal (F(1,14)=27.73, p<0.0001). FIG. 17C summarizes rates of oligodendrocyte replacement prior to and during the rehearsal phase as a function of prior training (“learn” in FIG. 17A) for cuprizone-treated mice, while FIG. 17D summarizes oligodendrocyte replacement rates as a function of prior training (“learn” in FIG. 17A) for cuprizone-treated mice over a 90-day training timeline. No effects of rehearsal on rate, inflection point, or asymptote of oligodendrocyte replacement were observed. FIG. 17E summarizes changes in activity during forelimb reach tasks for mice raised on cuprizone-containing and cuprizone-free diets. No effects of cuprizone on change in reaching behavior were observed between learning and rehearsal. FIG. 17F provides area plot of reach attempt outcomes in control and cuprizone-demyelinated mice. FIG. 17G summarizes peak success rates in forelimb reach tests during learning and rehearsal phases by mice raised on cuprizone-containing and cuprizone-free diets. Interaction effect between performance phase (learning vs. rehearsal) and drug (control vs. cuprizone) to predict success rate (F(1)=4.62, p=0.04). While control and cuprizone mice do not differ in success rate during pre-cuprizone learning, control mice perform significantly better during rehearsal relative to cuprizone-treated mice (Tukey's HSD, p=0.0004). Both cuprizone and cuprizone-treated mice have improved performance during rehearsal relative to learning (p=0.0001 and p<0.0001, respectively). FIG. 17H correlates peak success rate during mouse forelimb reach tests with peak oligodendrocyte loss during a cuprizone diet. No relationship between peak oligodendrocyte loss post-cuprizone and peak reaching success rate during rehearsal was observed. FIG. 17I provides asymptote of oligodendrocyte replacement as a function of rehearsal success for mice raised on cuprizone. No relationship between rehearsal success rate and asymptote of oligodendrocyte replacement was observed. *p<0.05, **p<0.01, ***p<0.001. Bars and errors represent Mean±SEM, points represent individual mice.

Mice in the “early-learning” group showed significant performance impairments relative to healthy controls and did not improve their reaching across the learning period, indicating a failure to acquire the reach task (FIG. 5E; FIGS. 16A-16L). While cuprizone did not alter overall reach attempts, the extent of demyelination was negatively related to performance (FIGS. 16A-16L). Motor deficits were temporally correlated to neuronal hyperexcitability in the forelimb region of motor cortex: firing rate was increased in demyelinated versus healthy mice in the first ten days post-cuprizone, coinciding with the entire early-learning period (FIGS. 5F-5G). Learning suppressed oligodendrocyte replacement rate by approximately 50% relative to untrained remyelinating mice (1.62±0.13% vs 3.21±0.59%, respectively; FIGS. 5B, 5H), resulting in a delayed inflection point of oligodendrocyte replacement (fifteen vs. nine days post-cuprizone, respectively; FIG. 5I). However, the asymptote of oligodendrogenesis did not differ between untrained and early-learning mice. The learning-induced suppression was less severe in remyelinating mice than in healthy controls (50% vs. 75%, respectively; see FIGS. 1A-1K) and an increase in oligodendrogenesis was not observed rate post-training (FIG. 5H). Success during learning was unrelated to the asymptote of oligodendrocyte replacement across mice (FIGS. 16A-16L). In sum, motor performance was impaired and motor cortex neurons were hyperexcitable following demyelination, and failing to learn the reach task provided no benefit to oligodendrogenesis during remyelination.

Since mice trained immediately following cuprizone cessation were unable to learn, mice were also trained ten days post-cuprizone (i.e., at about the half-maximum of the remyelination response; FIG. 3H). These “delayed-learning” mice showed no overall impairments in reaching performance (FIG. 5J) nor reaching attempts (FIGS. 16A-16L) relative to healthy mice. Again, it was found that neuronal hyperexcitability was temporally correlated to reaching success. While demyelinated mice were slightly less successful than healthy controls on the initial day of training (ten days post-cuprizone, when demyelinated mice still show motor cortex neuronal hyperexcitability; FIGS. 5K-5L), their success rates were indistinguishable from controls by the end of training (seventeen days post-cuprizone). Delayed-learners demonstrated a slight decrease in oligodendrogenesis rate during learning (˜30%) that was not statistically different from untrained demyelinated mice (FIGS. 5B, 5M). While the rate of oligodendrogenesis slowed by three weeks post-cuprizone in untrained mice, it did not in delayed-learners. The inflection point of oligodendrocyte replacement was therefore delayed in delayed-learners (thirteen vs. nine days post-cuprizone, respectively; FIG. 5N) and oligodendrocyte replacement plateaued substantially higher than in untrained mice (74.56+2.26% vs. 60.52+3.03%, respectively). Success during delayed-learning was not related to oligodendrocyte replacement (FIGS. 16A-16L). In sum, partial remyelination restored both neuronal function and the ability to learn the forelimb reach task, and motor learning following partial remyelination promoted long-term oligodendrogenesis.

To control for motor activity rather than motor learning, mice were trained pre-cuprizone administration and rehearsed the forelimb reach task post-cuprizone (FIGS. 17A-17I). Although demyelinated mice demonstrated performance deficits during rehearsal, rehearsal did not modulate any aspect of remyelination. Only learning the reach task (“delayed-learning”), but not attempting to learn it (“early-learning”) nor rehearsing it (“rehearsal”), promoted oligodendrogenesis post-cuprizone.

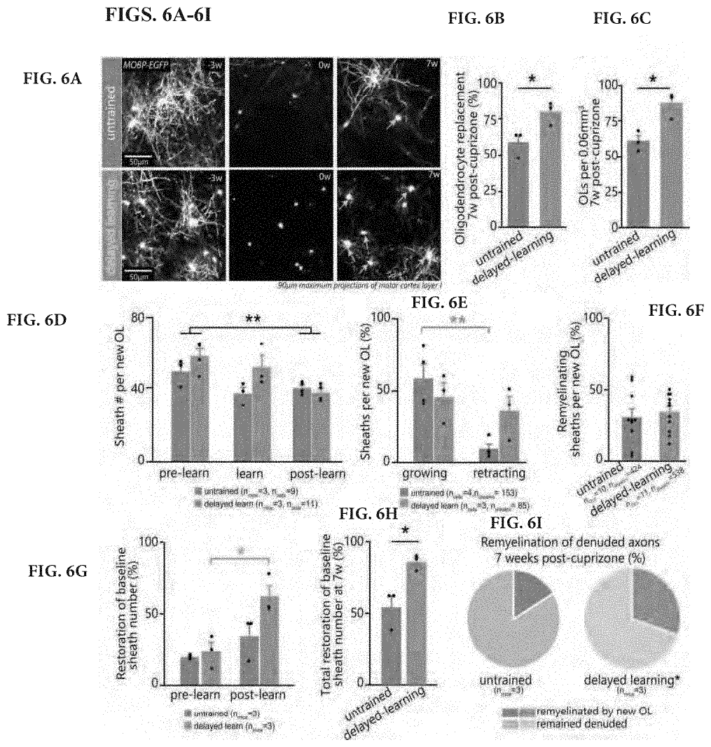

FIGS. 6A-6I illustrate that delayed motor learning can promote remyelination via new oligodendrocytes. FIG. 6A displays motor cortex images highlighting oligodendrocytes in the “untrained” and “delayed learning” mice outlined in FIG. 5A, as well as representative maximum-projections of superficial cortical oligodendrocytes (OLs) at baseline (left; −3 weeks), end of cuprizone diet (middle; 0 weeks), and following 7 weeks of remyelination (right) in untrained (top) and delayed-learning (bottom) mice. Yellow arrows designate new oligodendrocytes. FIG. 6B compares oligodendrocyte replacement 7 weeks post-cuprizone treatment for the “untrained” and “delayed learning” mice. FIG. 6C provides oligodendrocyte motor cortex densities (per 0.06 mm³) 7 weeks post-cuprizone treatment for the “untrained” and “delayed learning” mice, and illustrates that delayed-learners replace a greater proportion of lost oligodendrocytes (Student's t-test; t(3.92)=−2.99, p=0.04) and have a higher density of cortical oligodendrocytes than untrained mice (t(3.72)=−3.87, p=0.02) by 7-weeks post-cuprizone (points represent individual mice). FIG. 6D compares the number of sheaths per new oligodendrocyte prior to, during, and following the learning phase for the “untrained” and “delayed learning” mice. While new OLs have increased sheath numbers in first versus third week post-cuprizone _{(F5,15=5.14,} p=0.006; p=0.0038, Tukey's HSD), delayed-learning does not modulate this relationship (p=0.1; points represent individual OLs.) FIG. 6E details the number of sheaths for growing and retracting oligodendrocytes for the “untrained” and “delayed learning” mice, suggesting that delayed-learning modulates sheath dynamics (F3,10=6.65, p=0.0095). Sheaths on new OLs are more likely to grow than retract in untrained (p=0.007, Tukey's HSD) but not delayed-learning mice (p>0.8; points represent individual OLs.) FIG. 6F provides the number of remyelinating sheaths per new oligodendrocyte for the “untrained” and “delayed-learning” mice. Sheaths from new OLs are equally likely to remyelinate denuded axons in untrained and delayed-learning mice (Student's t-test; t(16.08)=−0.52, p=0.6; points represent individual OLs.) FIG. 6G, Population-level extrapolations suggest that delayed-learning modulates restoration of baseline sheath number (F3,8=7.80, p=0.0093; points represent mice). More sheaths are replaced after training in delayed-learning mice (Tukey's HSD; p=0.018). FIG. 6H, Population-level extrapolations suggest that delayed-learners restore a greater proportion of baseline sheath number 7 weeks post-cuprizone (Student's t-test; t(2.64)=−3.76, p=0.0407; points represent mice). FIG. 6I, Extrapolating sheath location probability to the population-level suggests that delayed-learners remyelinate a greater proportion of denuded axons than untrained mice (31% vs. 19%, respectively; Student's t-test; t(3.14)=−5.07, p=0.013). *p<0.05, **p<0.01, ***p<0.001, bars and error bars represent mean±SEM.

By seven weeks post-cuprizone, delayed-learning mice replaced over 20% more oligodendrocytes and had over 40% greater density of oligodendrocytes in layers I-III of motor cortex than age-matched, demyelinated, untrained mice (79.24±4.56% vs 58.43±5.26%, and 86.67±5.36 vs. 60.67±4.06, respectively; FIGS. 6A-6C). Delayed-learning did not modulate the number of sheaths per new oligodendrocyte (FIG. 6D) but did increase the proportion of sheaths that retracted over time (FIG. 6E), similar to observations in healthy mice (FIGS. 1I-1K). Sheaths from new oligodendrocytes were equally likely to remyelinate denuded axons in both untrained and delayed-learning mice (FIG. 6F). Using mean sheath number per new oligodendrocyte per mouse, restoration of baseline sheath number and remyelination of denuded axons were modeled to a population level. While untrained and delayed-learning mice replaced similar proportions of baseline sheath number prior to behavioral intervention (19.59±0.97% and 24.05±6.54%, respectively; FIG. 6G), delayed-learners replaced almost twice as many lost sheaths as untrained mice post-training (62.22±8.12% versus 34.72±8.84%) due to prolonged oligodendrogenesis (FIG. 5N and FIGS. 6A-6C). As a result, it was project that by seven weeks post-cuprizone, delayed-learners would have replaced almost 90% of their baseline sheath number, versus only 54% in untrained mice (FIG. 6H). Increased sheath generation by delayed-learners resulted in a predicted two-fold increase in remyelination of denuded axons relative to untrained mice (30.19±1.33% versus 16.38±2.37%, respectively; FIG. 6I).

Longitudinal in vivo two-photon imaging of the forelimb motor cortex throughout the learning of a forelimb reach task revealed that learning transiently suppressed oligodendrogenesis but subsequently increased oligodendrocyte generation, OPC differentiation, and retraction of pre-existing myelin sheaths. It was found that cuprizone-mediated demyelination induced ˜90% oligodendrocyte loss and neuronal firing rate abnormalities in the forelimb region of motor cortex, as well as deficits in motor performance. Motor learning only occurred following partial remyelination and restoration of neuronal function, and resulted in greater oligodendrocyte and myelin sheath replacement. Additionally, motor learning enhanced the ability of surviving oligodendrocytes to participate in remyelination via the generation of new sheaths. These results demonstrate that motor learning can improve remyelination via cortical oligodendrogenesis and myelin sheath formation by surviving oligodendrocytes.

OPC differentiation was unaffected during this suppression, suggesting that learning may temporarily decrease the survival and integration of differentiated OPCs as mature myelinating oligodendrocytes, in line with previous work in the developing CNS. These nuanced effects may be apparent due to the high intra-individual resolution and regional specificity, whereas previous studies have not parcellated motor cortex based on function. It is possible that location-specific cues suppress the integration of new oligodendrocytes to prevent aberrant myelination during learning, or that metabolic demand imposed by network plasticity during learning may deplete the resources required for the generation and integration of adjacent oligodendrocytes. How these learning-induced changes are communicated to the oligodendrocyte lineage cells remains undefined. Axons form synapses with local OPCs, and neuronal activity can modulate OPC proliferation and differentiation within both the healthy CNS and demyelinated regions. This communication may be mediated by the effects of brain-derived neurotrophic factor on both activity-dependent synaptic modulation and oligodendrocyte maturation and myelination.

### Example 10

### Motor Learning Promotes the Participation of Pre-Existing Mature Oligodendrocytes in Remyelination

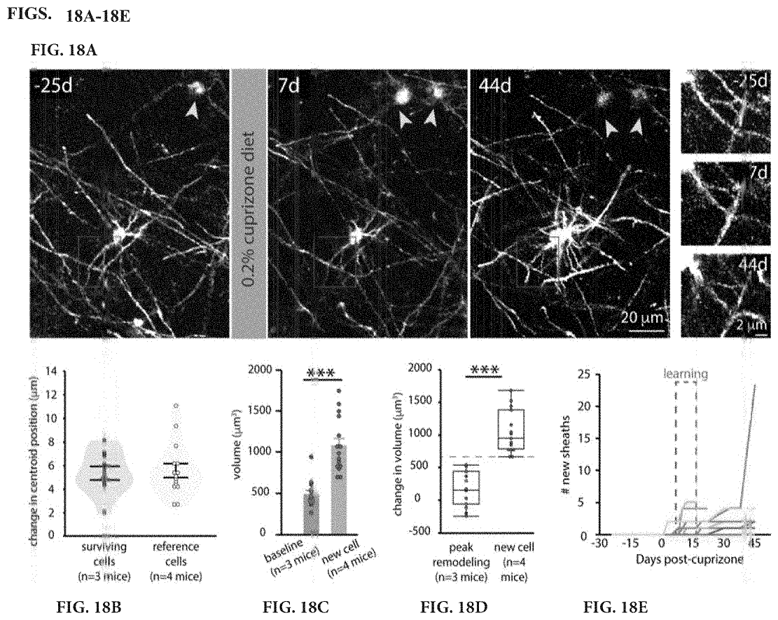

This example demonstrates a role of pre-existing mature oligodendrocytes in motor learning-mediated remyelination. To determine the contribution of pre-existing mature oligodendrocytes to remyelination, longitudinal in vivo imaging and semi-automated tracing were used to reconstruct myelin sheaths and connecting processes to the oligodendrocyte cell body (FIGS. 12A-12F). Myelin sheaths of individual oligodendrocytes were tracked throughout cuprizone-mediated demyelination and remyelination (FIGS. 18A-18E). Oligodendrocyte survival was variable between mice but did not differ in untrained and delayed-learning groups (12.29±7.32% vs. 20.84±6.60%; FIGS. 18A-18E).

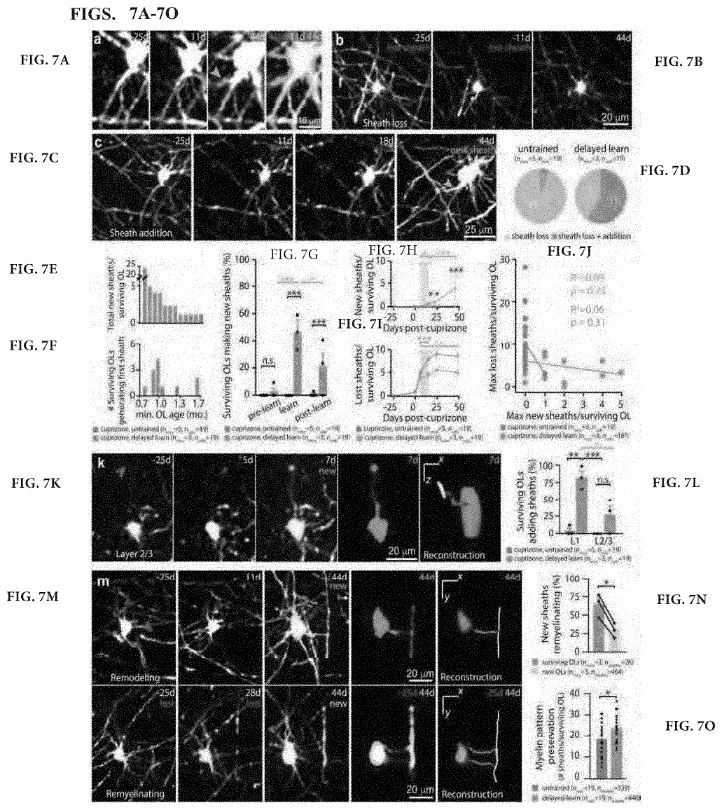

FIGS. 7A-7O illustrates that delayed motor learning can stimulate surviving mature oligodendrocytes to contribute to remyelination. FIG. 7A provides mouse motor cortex images highlighting surviving oligodendrocytes prior to (leftmost), 11 days following (second from left), and 44 days following (second from right) cuprizone treatment for “untrained” mice Identification of surviving oligodendrocytes (OLs) via conserved processes. The cyan arrow indicates new process on the same oligodendrocyte in a and c (cyan arrow). FIG. 7B provides mouse motor cortex images highlighting surviving oligodendrocytes prior to (leftmost), 11 days following (second from left), and 44 days following (second from right) cuprizone treatment for “untrained” mice. Pink highlights loss by OLs, while green indicates sheath generation. FIG. 7C displays a manually resliced version of the rightmost panel of FIG. 7B to show sheath and process connecting to cell body. FIG. 7D compares proportions of oligodendrocytes exhibiting only sheath loss to oligodendrocytes exhibiting both sheath loss and sheath growth in the “untrained” and “delayed-learning” mice. FIG. 7E provides the numbers of new sheaths in surviving oligodendrocytes in the “untrained” and “delayed-learning” mice. FIG. 7F summarizes the numbers of surviving oligodendrocytes based on age in the “untrained” and “delayed-learning,” indicating the number of sheaths generated per surviving OL and minimum possible OL age at time of sheath generation (assuming age 0 at imaging onset). FIG. 7G provides the numbers of surviving oligodendrocytes making new sheaths during pre-learning, learning and post-learning periods for “delayed-learning” mice and for “untrained” mice, showing that delayed-learning can modulate surviving OL sheath production (F2,51=9.30, learning (p=0.019), resulting in elevated generation relative to untrained mice both during (p<0.0001) and after learning (p=0.026). FIG. 7H summarizes the number of new sheaths per surviving oligodendrocyte as a function of time post-cuprizone treatment for the “untrained” and “delayed-learning” mice, thereby demonstrating that learning can modulate cumulative new sheaths on surviving OLs _{(F7,618=12.96,} p<0.0001). Delayed-learning increases new sheaths relative to baseline (p=0.019) and relative to untrained mice both during (p=0.028) and after (p=0.033) learning. Sheath number increases up to 4 weeks post-learning (p<0.0001). FIG. 7I summarizes the number of lost sheaths per surviving oligodendrocyte as a function of time post-cuprizone treatment for the “untrained” and “delayed-learning” mice, and showing that learning can modulate cumulative lost sheaths on surviving OLs _{(F7,611=7.04,} p<0.0001). Sheath loss initially increases in untrained and delayed-learning mice (p<0.0001 and p<0.0001, respectively) then ceases in delayed-learning (p>0.9) but not untrained mice (p<0.0001). FIG. 7J provides the maximum number of lost sheaths as a function of new sheaths in surviving oligodendrocytes in the “untrained” and “delayed-learning” mice. No relationship was observed between sheath loss and gain (*single outlier removed for analysis). FIG. 7K provides mouse motor cortex images highlighting surviving oligodendrocytes prior to (leftmost), 5 days following (second from left), and 7 days following (middle) cuprizone treatment for “untrained” mice. FIG. 7L compares the percent of surviving oligodendrocytes which added sheaths in different image slices for the “untrained” and “delayed-learning” mice. Learning can increase sheath generation by surviving OLs in both L1 and L/3 relative to controls (F1,6=7.05, p=0.038; p=0.0019 and p=0.0016, respectively), though generation was heightened within L1 versus L2/3 (p=0.044). Pink arrows point to location of junction between new sheath and surviving OL process. Relevant sheaths pseudo-colored. FIG. 7M provides mouse motor cortex images of newborn oligodendrocytes in previously unmyelinated (top, “Remodeling”) and myelinated (bottom, “Remyelinating”) areas at different timepoints relative to cuprizone treatment. The fourth and fifth columns from the left provide reconstructions of myelin sheath growth, with green and yellow indicating new myelin and purple indicating lost myelin. FIG. 7N summarizes the development of new myelin sheaths in previously myelinated areas (“Remyelinating”) for surviving and new oligodendrocytes. Three weeks post-cuprizone, new sheaths from surviving OLs were more likely to remyelinate denuded axons than sheaths from new OLs (t(2)=7.28, p=0.018). FIG. 7O compares the number of sheaths per surviving oligodendrocyte in the “untrained” and “delayed-learning” mice. Surviving OLs in delayed-learning mice were shown to contribute more sheaths to the original pattern of myelination (via maintenance and addition) than untrained mice (t(35)=−2.25, p=0.031). *p<0.05, **p<0.01, ***p<0.001, bars and error bars represent mean±SEM.

FIGS. 18A-18E overviews identification of oligodendrocytes that survive demyelination. FIG. 18A provides representative images outlining the methodology for following surviving oligodendrocytes over time. Single plane images of the same oligodendrocyte at baseline (−25 d), one week after demyelination (7 d), and six weeks after demyelination (44 d). Red boxes highlight one example of the same oligodendrocyte processes lasting for the duration of the study. Single plane image of the same oligodendrocyte at baseline (−25 d), one week after demyelination (7 d), and six weeks after demyelination (44 d). Red boxes highlight one example of the same oligodendrocyte processes lasting for the duration of the study. The maintenance of the spatial relationship between the oligodendrocyte of interest and other oligodendrocytes in the field of view (yellow arrowheads) provide further confirmation of oligodendrocyte identity. A new cell that appears at 7 d. FIG. 18B provides changes in centroid position of reference oligodendrocytes within the z-stack and surviving cell bodies from baseline to day of peak remodeling—i.e. the day where the largest number of sheaths were added by a given oligodendrocyte. FIG. 18C summarizes volumes (μm³) for oligodendrocytes pre-cuprizone treatment and for new cells post-cuprizone treatment. Surviving oligodendrocytes at baseline were significantly smaller than new oligodendrocytes (t(21.91)=−5.81, p<0.0001, Student's t-test). FIG. 18D summarizes changes in volume (μm³) for oligodendrocytes pre-cuprizone treatment and for new cells post-cuprizone treatment. Change in volume of surviving oligodendrocytes from baseline to peak remodeling was significantly smaller than the volume of new oligodendrocytes (t(23.88=−7.59, p<0.0001). FIG. 18E provides the number of new myelin sheaths on multiple oligodendrocytes, thereby highlighting dynamics of sheath addition over time. Each line represents an individual oligodendrocyte. *p<0.05, **p<0.01, ***p<0.001. Bars and errors represent Mean±SEM, box plots represent Median and IQR.

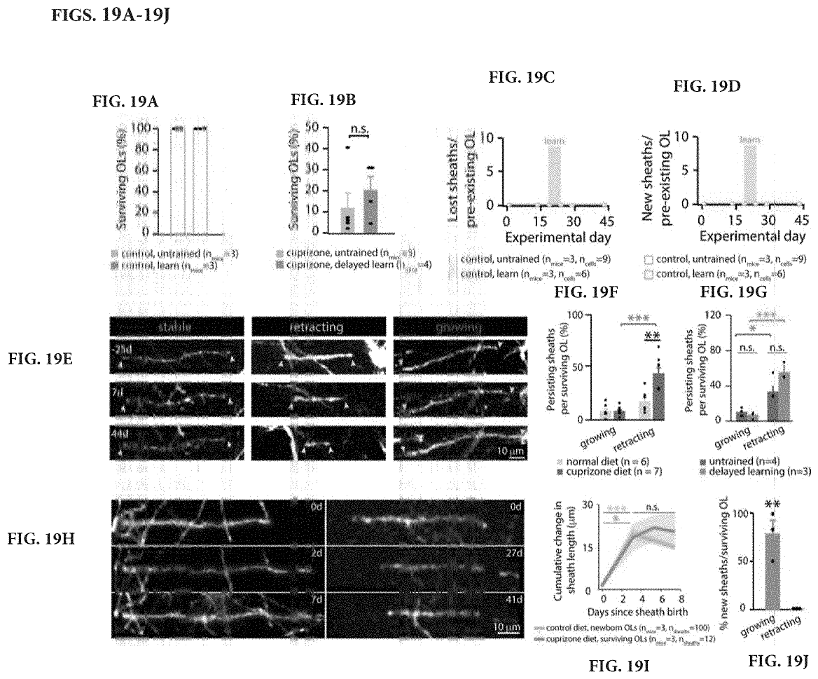

FIGS. 19A-19J Dynamics of pre-existing and newly-generated myelin sheaths from surviving oligodendrocytes. FIG. 19A summarizes the percentage of surviving oligodendrocytes in forelimb reach task trained and forelimb reach task untrained mice raised on cuprizone-free diets, showing that no oligodendrocytes were lost in healthy mice. FIG. 19B summarizes the percentage of surviving oligodendrocytes in forelimb reach task trained and forelimb reach task untrained mice raised on cuprizone-containing diets. No difference in percent of oligodendrocytes (OLs) surviving demyelination in untrained and delayed learning groups was observed (Wilcoxon Rank-Sum, p>0.5). FIG. 19C summarizes the number of lost myelin sheaths on pre-existing oligodendrocytes in forelimb reach task trained and forelimb reach task untrained mice raised on cuprizone-free diets. FIG. 19D summarizes the number of new myelin sheaths on pre-existing oligodendrocytes in forelimb reach task trained and forelimb reach task untrained mice raised on cuprizone-free diets. No sheaths were lost (FIG. 19C) nor generated (FIG. 19D) on mature oligodendrocytes in healthy trained or untrained conditions. FIG. 19E provides images of stable, retracting, and growing oligodendrocytes at different timepoints along the training course, highlighting the behavior of pre-existing myelin sheaths that persist throughout study. Relevant sheaths are pseudo colored. FIG. 19F. summarizes the number of persisting sheaths per surviving growing or retracting oligodendrocyte for forelimb reach task trained mice raised on cuprizone-containing diets and cuprizone-free diets. Three weeks into remyelination, sheath retraction was significantly increased (F(3,22)=18.65, p<0.0001) when compared to age-matched controls (Tukey's HSD, p=0.0006) and when compared to the percent of sheaths growing in cuprizone-treated mice (p<0.0001). FIG. 19G summarizes the number of persisting sheaths per surviving growing or retracting oligodendrocyte for forelimb reach task trained and forelimb reach task untrained mice raised on cuprizone-containing diets. No effect of delayed learning on sheath dynamics was observed during remyelination. Sheaths retracted more than they grow in both untrained (p=0.016) and delayed learning mice (p=0.0003). FIG. 19H provides images showing maximum projection of new sheaths generated after cuprizone exhibiting growth (pseudo colored green, left) and retraction (pseudo colored red, right). FIG. 191 summarizes cumulative changes in myelin sheath length for oligodendrocytes as a function of days since birth. New myelin sheaths changed in length in the week following their generation, whether they were from new oligodendrocytes (control: F(3,302)=47.94, p<0.0001) or from surviving oligodendrocytes after cuprizone-demyelination (cuprizone diet: F(3,29)=5.31, p=0.0049). Sheaths in both control and cuprizone treatment stabilize their length within 3 days of sheath birth (d0 vs. d3, p<0.0001 in control and p=0.028 in cuprizone; Tukey's HSD). Line and shading represent mean and SEM. FIG. 19J summarizes the number of new sheaths for growing and retracting oligodendrocytes. Sheaths from pre-existing oligodendrocytes grew more often than they retracted the first three days post-generation (Wilcoxon Rank-Sum, p=0.0029). *p<0.05, **p<0.01, ***p<0.001. Bars and errors represent Mean±SEM.

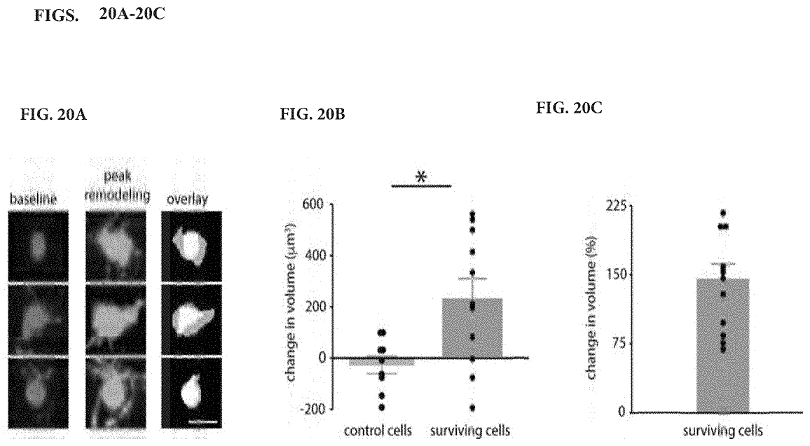

FIGS. 20A-20C outlines surviving oligodendrocyte cell soma volume changes during remyelination. FIG. 20A provides images highlighting maximum projection of surviving oligodendrocyte cell bodies at baseline (left, magenta), peak remodeling (middle, cyan), and overlaid (right), with a scale bar showing 10 μm. FIG. 20B summarizes changes in oligodendrocyte cell body volume in mice subjected to the “delayed-learning” regimen, Oligodendrocytes in normal untrained mice displayed little change in cell body volume throughout the study, from baseline (0 d) to 43 d. Surviving cells in delayed learning mice showed dramatic increase in cell soma volume from baseline to day of peak remodeling when compared to oligodendrocytes in normal untrained mice (t(12.24)=2.56, p=0.025, Student's t-test). FIG. 20C provides percent change in volume between baseline and day of sheath addition for surviving cells engaging in remodeling. *p<0.05, **p<0.01, ***p<0.001. Bars and errors represent Mean±SEM.

After three weeks of cuprizone treatment in untrained mice, all surviving oligodendrocytes experienced sheath loss, and in rare instances ( 1/19) oligodendrocytes added a new sheath (FIGS. 7A-7D). While pre-existing myelin sheaths in healthy mice rarely remodeled (see FIGS. 1A-1K), cuprizone treatment increased pre-existing sheath retraction in surviving oligodendrocytes (17.0±4.22% vs. 43.8±5.95%; FIGS. 19A-19J). Delayed-learning did not affect the degree of remodeling in pre-existing myelin sheaths during remyelination (FIGS. 19A-19J), but dramatically increased the number of pre-existing oligodendrocytes that generated new myelin sheaths (FIGS. 7D, 7E). Sheath generation in preexisting oligodendrocytes followed a similar time course to new oligodendrocytes, with sheaths growing during the first three days post-generation before stabilization (FIGS. 19A-19J). In healthy mice, pre-existing oligodendrocytes were never observed generating new sheaths (FIGS. 19A-19J), however, in delayed-learning mice, pre-existing oligodendrocytes were able to generate sheaths even 1.7 months after the onset of imaging, suggesting that the ability to generate myelin sheaths is an extended property of oligodendrocytes (FIG. 7F).

The generation of new sheaths from pre-existing oligodendrocytes was temporally correlated with the onset of forelimb reach training. The number of pre-existing oligodendrocytes generating new sheaths increased by over 40% during learning and persisted in the following weeks (FIG. 7G). As such, delayed-learners had a higher cumulative number of new sheaths generated by surviving oligodendrocytes than untrained mice both during and after learning (FIG. 7H). Myelin sheath loss stagnated in surviving oligodendrocytes after the onset of learning, in contrast to sheath loss in untrained mice which continued for two weeks post-cuprizone (FIG. 7I). The number of lost myelin sheaths was unrelated to sheaths generated on individual oligodendrocytes (FIG. 7J).

As with oligodendrogenesis following learning, sheath addition by pre-existing oligodendrocytes was higher in layer I versus layer II/III of cortex (FIGS. 7K-7L). Surviving oligodendrocytes formed new myelin sheaths on both denuded and previously unmyelinated axons (FIG. 7M). A significantly larger proportion of surviving oligodendrocyte sheaths remyelinated denuded axons relative to newly generated oligodendrocytes (FIG. 7N). The combination of learning-induced cessation of sheath loss and new sheath generation from surviving oligodendrocytes resulted in greater maintenance of the original myelination pattern in delayed learning mice relative to untrained mice (FIG. 7O). Pre-existing oligodendrocytes engaging in new myelin sheath deposition showed an increase in overall cell body volume of 141±15% (FIGS. 20A-20C). These findings indicate that, following demyelination, motor learning specifically enhances the ability of pre-existing oligodendrocytes to generate additional myelin and maintain pre-existing sheaths.

### Example 11

Vagus Nerve Stimulation Paired with Motor Learning

This example explores effects of vagus nerve stimulation in oligodendrogenesis and remyelination. To investigate the role of paired-VNS in remyelination, VNS was applied in a cuprizone-induced demyelination model during learning of a novel forelimb reach task and tracked the fate of oligodendrocytes (OLs) with longitudinal imaging over primary motor cortex (M1). To enable longitudinal imaging, craniotomy was applied over M1 on six- to eight-week old C57BL/6N MOBP-EGFP mice. To induce cortical demyelination, ten-week old mice with cranial windows were put on 0.2% cuprizone diet for three weeks. Mice can receive either invasive stimulation by implanting electrodes on the left vagus nerve or transcutaneous stimulation by putting stimulation device on the ear.

In these experiments, stimulation was applied directly to the cervical vagus nerve using an implanted stimulation cuff. After cuprizone diet, both paired-VNS and control group learned a novel forelimb reach task during 20-minute sessions over seven days. During training, stimulation (0.2-0.6 mA, 100 μs pulse, 30 Hz) was applied on trial where the mouse successfully brought the food pellet back to the training box. A “VNS_alone” group received similar stimulation in the training box as paired-VNS for seven days without the forelimb reach task involved. Images were taken from the first day of cuprizone diet until four-weeks post cuprizone diet.

The same forelimb task was re-introduced to mice from paired-VNS and control group at 2.5-months post cuprizone to test their ability of motor performance (rehearsal phase). Due to the variations in total OL loss and its subsequent effects on OL gain, percentage of OL replacement, which defines the ratio of OL gain to maximum OL loss, was used to indicate remyelination, as described in previous study.

A logistic 3P prediction model was used to fit sigmoidal curves that is bound between 0 (baseline) and an asymptote value of oligodendrocyte accumulation (either loss, gain or replacement). When comparing between two groups, either two-tailed student's t-test or Wilcoxon test was performed, depending on if the dataset satisfies the normality tests. When comparing within groups, paired student's t-test was performed. Two-stage step-up method of Benjamini, Krieger and Yekutieli was performed for multiple comparisons. A restricted maximum likelihood approach (REML) with Tukey's honestly significant difference (HSD) post-hoc test was used for statistical mixed modeling. For this modeling, “Mouse ID” was assigned as random effect.

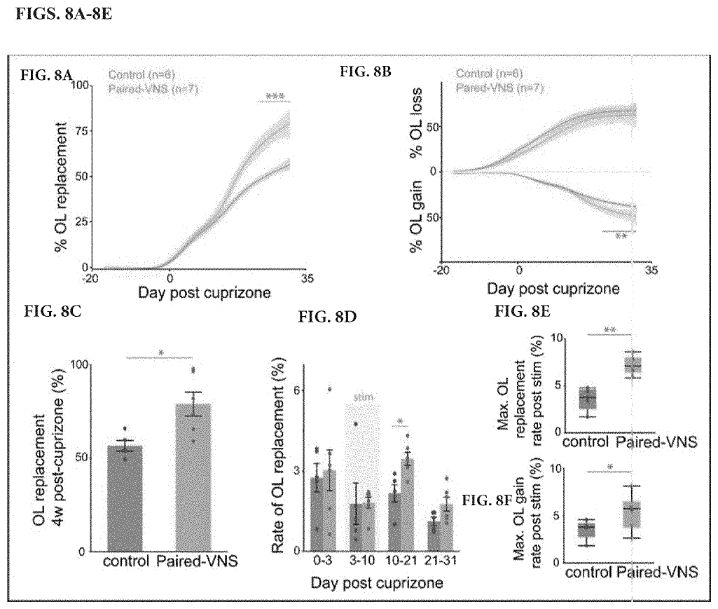

FIGS. 8A-8F illustrate that paired-VNS improves remyelination with a higher replacement rate post stimulation. FIG. 8A, illustrates a significantly improved asymptote replacement in stim group compared to control (Student's t-test; t(11)=5.624, P=0.0002). FIG. 8B illustrates an increase in asymptote OL gain in stim group versus control (t(11)=3.290, P=0.0072) with no differences in asymptote OL loss (t(11)=1.125, P=0.28). FIG. 8C provides the OL replacement at four-weeks post cuprizone, and illustrates that OL replacement was higher in stim group compared to control (Student's t-test, t(9)=3.039, P=0.014). FIG. 8D provides OL replacement rates, and illustrates that OL replacement was higher in stim group than control at 10 days post cuprizone (Multiple unpaired t-tests; t(9)=3.496, P=0.021). FIG. 8E illustrates maximum OL replacement rate in the test and control groups. FIG. 8F illustrates that maximum OL gain rate post stimulation was highest in the stim group (Student's t-test; Maxi OL replacement rate: t(9)=4.511, P=0.0016; Maxi OL gain rate: t(9)=2.301, P=0.047).

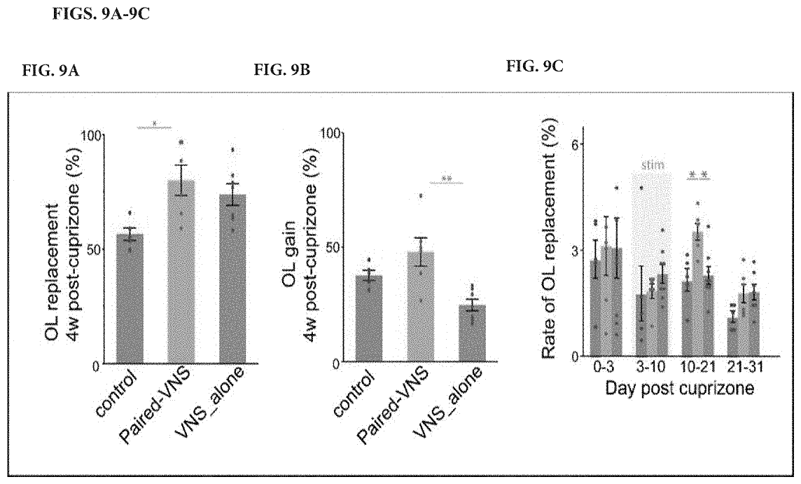

FIGS. 9A-9C demonstrates that stimulation during learning brings larger improvement on remyelination. FIG. 9A illustrates that only the paired-VNS treatment significantly increased the OL replacement at four-weeks post cuprizone compared to control (P=0.020, Turkey's HSD). FIG. 9B illustrates that OL gain at four-weeks post cuprizone was significantly less in VNS_alone (P=0.003, Turkey's HSD). FIG. 9C outlines that only paired-VNS significantly increased the rate of OL replacement (P=0.01 and P=0.02 respectively, Turkey's HSD).

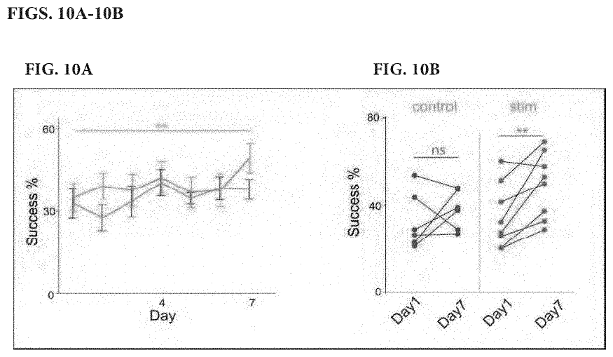

FIGS. 10A-10B illustrate that paired-VNS may enhance the ability to learn in the long term. FIG. 10A illustrates that behavioral performance was only improved in stim group across days (ctrl: P=0.1 vs. paired-VNS: P=0.009, Turkey's HSD). FIG. 10B illustrates that paired-VNS group performed better at day 7 compared to day 1 (Paired t-test; Ctrl: t(5)=0.872, P=0.42; Paired-VNS: t(6)=3.486, P=0.013).

Paired-VNS enhanced remyelination and may have improved learning ability in the long term compared to surgical control. The asymptote OL replacement was significantly improved in paired-VNS group (ctrl=57.28 ±3.08% vs. paired-VNS=83.55±3.42%) (FIG. 8A) with an increase in asymptote OL gain (ctrl=38.65±2.54% vs. paired-VNS=50.82±2.65%) and no difference in asymptote OL loss (ctrl=68.62±2.74% vs. paired-VNS=64.13±2.85%) (FIG. 8B). OL replacement was enhanced by 4-weeks post cuprizone (ctrl=56.43±2.77% vs. paired-VNS=79.98±6.63%) (FIG. 8C). Moreover, this enhancement occurred post stimulation instead of during stimulation, indicated by a higher replacement rate at day 10-21 post cuprizone (FIG. 8D). Paired-VNS almost doubled the maximum OL replacement rate (ctrl=3.669±0.543% vs. paired-VNS=7.024±0.506%) (FIG. 8E) as well as the maximum OL gain rate (ctrl=2.430±0.350% vs. paired-VNS=4.427±0.731%) post stimulation (FIG. 8F), suggesting that paired-VNS improved their ability of remyelination. Although VNS_alone exhibited slightly higher OL replacement by 4-weeks post cuprizone compared to control as paired-VNS (ctrl=56.43±2.77% vs. paired-VNS=79.98±6.63% vs. VNS_alone=73.82±4.73%) (FIG. 9A), the OL gain by 4-weeks post cuprizone was significantly smaller than paired-VNS (paired-VNS=47.96±6.23% vs. VNS_alone=24.73±2.56%) (FIG. 9B). In addition, only paired-VNS increased the replacement rate post stimulation (FIG. 9C), suggesting that VNS_alone illustrates smaller, if any, effects on remyelination and pairing stimulation with learning was much more beneficial to remyelination. For their motor performance at rehearsal phase, where the difference of remyelination existed between groups, success rate was calculated. Only paired-VNS group exhibited an improved performance across days (FIG. 10A) and did significantly better at day 7 compared to day 1 (FIG. 10B), implying that VNS provided better ability to learn in the long term.

### Methods

Animals: Male and female mice used in these experiments were kept on 14 h light/10 h dark schedule with ad libitum access to food and water, aside from training-related food restriction (see Forelimb Reach Training). All mice were randomly assigned to conditions and were precisely age-matched (±5 days) across experimental groups. NG2-mEGFP (Jackson stock #022735) and congenic C57BL/6N MOBP-EGFP (MGI:4847238) lines, which have been previously described, were used for two-photon imaging. Wild-type C57\B6N Charles River wild-type mice were used in electrophysiological experiments.

All publications and patent applications cited in this specification are herein incorporated by reference as if each individual publication or patent application were specifically, and individually, indicated to be incorporated by reference.

All of the COMPOSITIONS and METHODS disclosed and claimed herein can be made and executed without undue experimentation in light of the present disclosure. While the COMPOSITIONS and METHODS have been described in terms of preferred embodiments, it will be apparent to those of skill in the art that variation can be applied to the COMPOSITIONS and METHODS and in the steps or in the sequence of steps of the METHODS described herein without departing from the concept, spirit and scope of the invention. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the invention as defined by the appended claims.

1. A method for reducing the progression of, preventing, and/or reducing demyelination in a subject having a demyelination disease, disorder, or condition, the method comprising having the subject perform at least one motor learning task.

2. The method according to claim 1, wherein the motor learning task is performed after the subject has suffered a demyelination event.

3. The method according to claim 2, wherein the demyelination event in the subject occurred within about 1 month to about 3 months from the time of having the subject perform at least one motor learning task.

4. The method according to claim 1, further comprising stimulating the vagus nerve of the subject.

5. The method according to claim 4, wherein stimulating the vagus nerve comprises stimulating the vagus nerve before, at the same time of, or after having the subject performs at least one motor learning task.

6. The method according to claim 5, wherein stimulating the vagus nerve comprises stimulating the vagus nerve at the same time the subject performs the at least one motor learning task.

7.-8. (canceled)

9. The method according to claim 1, wherein at least one motor learning task comprises at least one of a fine motor skill or a gross motor skill.

10. The method according to claim 4, wherein stimulating the vagus nerve of the subject comprises vagus nerve stimulation (VNS) either through an invasive implanted stimulation device or through a non-invasive stimulation device.

11. (canceled)

12. The method according to claim 4, wherein stimulating the vagus nerve comprises electrical stimulation.

13. The method according to claim 12, wherein the electrical stimulation comprises using about 0.05 to about 100 milliamperes or about 0.5 to about 20 milliamperes electrical current.

14. The method according to claim 12, wherein the electrical stimulation comprises about 1 microsecond (μs) to about 1 millisecond (ms) or about 10 to about 100 microsecond (μs) electrical pulses.

15. The method according to claim 12, wherein the electrical stimulation comprises a frequency of about 0.3 to about 3000 Hz or about 3 to about 300 Hz.

16. The method according to claim 1, further comprising administering a pharmaceutical composition to the subject and reducing the progression of, preventing, and/or reducing demyelination in a subject.

17. The method according to claim 1, wherein the subject comprises a subject having at least one of multiple sclerosis (MS), Alzheimer's disease, Parkinson's disease, Huntington's disease, Amyotrophic lateral sclerosis (ALS), chronic inflammatory demyelinating polyneuropathy (CIDP), Batten disease, acute disseminated encephalomyelitis (ADEM), acute optic neuritis (AON), transverse myelitis, Neuromyelitis optica spectrum disorders (NMO); cranial neuropathies, autonomic neuropathies or other neuropathy causing demyelination, traumatic brain injury (TBI), side effects of a brain injury, accident or a concussion.

18. The method according to claim 17, wherein the subject comprises a subject having at least one side effect of a brain injury, accident or a concussion.

19. The method according to claim 17, wherein the at least one motor learning task is first performed at least 7 days, or at least 14 days, or at least 28 days after the TBI, accident, or concussion.

20. The method according to claim 1, wherein performing at least one motor learning task comprises performing at least one motor learning task multiple times per day, twice per day, daily, every other day, a couple times a week or other appropriate regimen.

21.-23. (canceled)

24. The method according to claim 1, further comprising restricting the caloric intake of the subject.

25. A method of promoting remyelination in a subject having a demyelination condition or disease comprising having the subject perform at least one motor learning task and stimulating the vagus nerve of the subject and promoting remyelination in the subject.

26. (canceled)
